(19) United States
(12) Patent Application Publication
(10) Pub. No.: US 2012/0027793 A1
(43) Pub. Date: Feb. 2, 2012

(54) COMPOSITIONS COMPRISING CHLAMYDIA ANTIGENS

(75) Inventors: Robert C. Brunham, Vancouver (CA); Leonard J. Foster, Vancouver (CA)

(73) Assignee: British Columbia Cancer Agency Branch, Vancouver (CA)

(21) Appl. No.: 13/147,092

(22) PCT Filed: Jan. 29, 2010
(86) PCT No.: PCT/CA10/00135

§ 371 Date: Oct. 17, 2011

Related U.S. Application Data
(60) Provisional application No. 61/202,104, filed on Jan. 29, 2009.
(60) Provisional application No. 61/202,943, filed on Apr. 22, 2009.

(51) Int. Cl. A61K 39/118; A61P 31/04; A61P 37/04

(52) U.S. Cl. 424/190.1; 424/263.1

(57) ABSTRACT

There is provided a composition for inducing an immune response to a Chlamydia species in a subject, the composition comprising one or more than one polypeptides selected from the group consisting of ribosomal peptide L/RplF, ribosomal protein L6 (RplF), PmpG protein, PmpG-1 peptide, PmpF protein, PmpE/F-2 family F2, glyceraldehyde 3-phosphate dehydrogenase and major outer membrane protein (MOMP), and the use of said composition to treat Chlamydia infections.

This application claims priority benefit of U.S. Provisional applications 61/202,104 filed Jan. 29, 2009 and 61/202,943, filed Apr. 22, 2009, the contents of which are herein incorporated by reference.

### TECHNICAL FIELD

The present invention relates to the field of immunology, and immunostimulatory agents. More specifically, the present invention relates to compositions comprising Chlamydia antigens; the compositions may be useful for inducing an immune response to a Chlamydia spp.

### BACKGROUND

C. trachomatis includes three human biovars: trachoma (serovars A, B, Ba or C), urethritis (serovars D-K), and lymphogranuloma venereum (LGV, serovars L1, 2 and 3). C. trachomatis is a obligate intracellular pathogen (i.e. the bacterium lives within human cells) and can cause numerous disease states in both men and women. Both sexes can display urethritis, proctitis (rectal disease and bleeding), trachoma, and infertility. The bacterium can cause prostatitis and epididymitis in men. In women, cervicitis, pelvic inflammatory disease (PID), ectopic pregnancy, and acute or chronic pelvic pain are frequent complications. C. trachomatis is also an important neonatal pathogen, where it can lead to infections of the eye (trachoma) and pulmonary complications.

Worldwide Chlamydia trachomatis is responsible for over 92 million sexually transmitted infections and 85 million ocular infections annually. Public health programs have targeted C. trachomatis as a major problem because of the ability of the organism to cause long term sequelae such as infertility, ectopic pregnancy and blindness. In developed countries, public health measures to prevent and control Chlamydia appear to be failing as case rates continue to rise and in developing countries efforts to control Chlamydia are not feasible using current approaches.

Immunity to Chlamydia is known to depend on cell-mediated immune (CMI) responses, especially Th1 polarized cytokine responses (Brunham et al., 2005). Antibodies appear to play a secondary role. Experience has shown that developing vaccines for intracellular pathogens that require protective CMI is more difficult than for pathogens that simply require protective antibody. Part of the problem has been the identification of antigens that induce protective CMI responses because protective antigens need to be presented to T cells by MHC molecules and identifying MHC-bound microbial epitopes has been difficult. Immunity to Chlamydia can be induced using whole inactivated C. trachomatis elementary bodies, but the vaccine efficacy was both incomplete and short lived. Additionally, breakthrough C. trachomatis infection in primate models resulted in more severe disease with worse inflammation post-vaccination. Other vaccine efforts have focused on subunit vaccines that comprise individual C. trachomatis antigens. The Chlamydia major outer membrane protein (MOMP) has been evaluated as a vaccine candidate in primate models, yet the MOMP-based vaccine only conferred marginal protection (Kari et al. Fourth Meeting of the European Society for Chlamydia Research, Aarhus, Denmark, 1-4 Jul. 2008).

Genomic-based approaches to identify candidate peptides, proteins, subunits or epitopes may provide an efficient method for identifying moieties with potential for use in a vaccine, particularly in the context of the well-studied mouse model. Li et al 2006 (Vaccine 24:2917-2927) used bioinformatic and PCR-based methods to produce cloned open reading frames (ORFs), which were in turn pool-inoculated into mice, with subsequent rounds of challenge and further screening to identify ORFs that demonstrated significant protection.

Making a vaccine for pathogens that require protective cell-mediated immunity (CMI) responses is more difficult than for pathogens which require protective antibody responses. Part of the problem has been the identification of individual antigens that induce protective CMI responses. Studies in animal models and during human infection have established that Chlamydia-specific CD4+ T cells producing gamma interferon (IFN-gamma) are critically involved in the clearance of a Chlamydia infection (Su et al. 1995 Infect Immun 63:3302-3308; Wang et al. 1999 Eur J Immunol 29:3782-3792). Design of an effective vaccine for a chlamydia infection may require the selection of antigens that effectively stimulates CD4+ Th1 cells.

Patents and patent applications disclosing nucleic acid or polypeptide compositions comprising full or partial MOMP sequences are described in, for example, U.S. Pat. No. 6,030,799, U.S. Pat. No. 6,696,421, U.S. Pat. No. 6,676,949, U.S. Pat. No. 6,464,979, U.S. Pat. No. 6,653,461 and US Patent Publication 2008/0102112.

Other Chlamydia sequences (nucleic acid and polypeptide) are described in, for example, U.S. Pat. No. 6,642,023, U.S. Pat. No. 6,887,843 and U.S. Pat. No. 7,459,524; and US Patent Publications 2005/0232941, 2009/0022755, 2005/0035296, 2006/0286128.

### SUMMARY OF THE INVENTION

The present invention relates to the field of immunology, and immunostimulatory agents. More specifically, the present invention relates to compositions comprising Chlamydia antigens; the compositions may be useful for inducing an immune response to a Chlamydia spp.

In accordance with one aspect of the invention, there is provided a composition for inducing an immune response to a Chlamydia species in a subject, the composition comprising one, or more than one polypeptide, selected from the group consisting of SEQ ID NO: 10, SEQ ID NO: 13, SEQ ID NO: 16, SEQ ID NO:17, SEQ ID NO: 20, SEQ ID NO: 21, SEQ ID NO: 22, SEQ ID NO: 23, SEQ ID NO: 27, SEQ ID NO: 28, SEQ ID NO: 31, SEQ ID NO: 42, SEQ ID NO: 43, SEQ ID NO: 45, SEQ ID NO: 46, and an excipient.

In accordance with another aspect of the invention, the composition may further comprise one, or more than one, of a polypeptide selected from the group consisting of PmpG, PmpF, PmpG-1, PmpE/F-2, and RplF.

In accordance with another aspect, the one, or more than one, polypeptide PmpG, PmpF, PmpG-1, PmpE/F-1, SEQ ID NO: 42, SEQ ID NO: 43, SEQ ID NO: 45, SEQ ID NO: 46, or RplF polypeptides are Chlamydia trachomatis polypeptides, or Chlamydia muridarum polypeptides. The composition may further comprise an adjuvant. The adjuvant may be dimethyldioctadecylammonium bromide and trehalose 6,6′-dibehenate (DDA/TDB) or AbISCO. The Chlamydia species may be C. trachomatis or C. muridarum.

In accordance with another aspect, the composition may further comprise a MOMP polypeptide, or a fragment or portion thereof. The fragment or portion thereof may comprise SEQ ID NO: 44 or SEQ ID NO: 47.

The immune response may be a cellular immune response.

In accordance with another aspect of the invention, there is provided a method of treating or preventing a Chlamydia infection in a subject, comprising administering to the subject an effective amount of a composition comprising one, or more than one, polypeptide selected from the group consisting of SEQ ID NO: 10, SEQ ID NO: 13, SEQ ID NO: 16, SEQ ID NO:17, SEQ ID NO: 20, SEQ ID NO: 21, SEQ ID NO: 22, SEQ ID NO: 23, SEQ ID NO: 27, SEQ ID NO: 28, SEQ ID NO: 31, SEQ ID NO: 42, SEQ ID NO: 43, SEQ ID NO: 45, SEQ ID NO: 46, PmpG, PmpF, PmpG-1, PmpE/F-2 and RplF, and an excipient. The Chlamydia infection may be in a lung or genital tract, or an eye, and may be a C. trachomatis infection. The composition may induce a cellular immune response, and may be administered intranasally, or by injection.

In accordance with another aspect of the invention, there is provided a composition for inducing an immune response in a subject, comprising one, or more than one, polypeptide selected from the group consisting of SEQ ID NO: 10, SEQ ID NO: 13, SEQ ID NO: 16, SEQ ID NO:17, SEQ ID NO: 20, SEQ ID NO: 21, SEQ ID NO: 22, SEQ ID NO: 23, SEQ ID NO: 27, SEQ ID NO: 28, SEQ ID NO: 31, SEQ ID NO: 42, SEQ ID NO: 43, SEQ ID NO: 45, SEQ ID NO: 46, PmpG, PmpF, PmpG-1, PmpE/F-2 and RplF, and an excipient.

In accordance with another aspect, the polypeptides PmpG, PmpF, PmpG-1, PmpE/F-1 or RplF, or fragments or portions thereof may be Chlamydia trachomatis polypeptides, or Chlamydia muridarum polypeptides.

In accordance with another aspect of the invention, there is provided a method of eliciting an immune response against Chlamydia trachomatis in a mammal, comprising administration of a therapeutically effective amount of a composition comprising one or more C. trachomatis polypeptides and an excipient. The polypeptides may be one, or more than one, of SEQ ID NO: 42, SEQ ID NO: 43 and SEQ ID NO: 44.

In accordance with another aspect of the invention, there is provided a use of a composition comprising one, or more than one, polypeptide selected from the group consisting of SEQ ID NO: 10, SEQ ID NO: 13, SEQ ID NO: 16, SEQ ID NO:17, SEQ ID NO: 20, SEQ ID NO: 21, SEQ ID NO: 22, SEQ ID NO: 23, SEQ ID NO: 27, SEQ ID NO: 28, SEQ ID NO: 31, SEQ ID NO: 42, SEQ ID NO: 43, SEQ ID NO: 45, SEQ ID NO: 46, PmpG, PmpF, PmpG-1, PmpE/F-2 and RplF, and an excipient. Chlamydia

In accordance with another aspect of the invention, there is provided a use of a composition comprising one, or more than one, polypeptide selected from the group consisting of SEQ ID NO: 10, SEQ ID NO: 13, SEQ ID NO: 16, SEQ ID NO:17, SEQ ID NO: 20, SEQ ID NO: 21, SEQ ID NO: 22, SEQ ID NO: 23, SEQ ID NO: 27, SEQ ID NO: 28, SEQ ID NO: 31, SEQ ID NO: 42, SEQ ID NO: 43, SEQ ID NO: 45, SEQ ID NO: 46 PmpG, PmpF, PmpG-1, PmpE/F-2 and RplF, and an excipient in the manufacture of a medicament for the treatment or prevention of a Chlamydia infection in a subject.

In accordance with another aspect of the invention, there is provided a method of treating or preventing a Chlamydia infection comprising administering an effective amount of a composition comprising one, or more than one, polypeptide selected from the group consisting of SEQ ID NO: 10, SEQ ID NO: 13, SEQ ID NO: 16, SEQ ID NO:17, SEQ ID NO: 20, SEQ ID NO: 21, SEQ ID NO: 22, SEQ ID NO: 23, SEQ ID NO: 27, SEQ ID NO: 28, SEQ ID NO: 31, SEQ ID NO: 42, SEQ ID NO: 43, SEQ ID NO: 45, SEQ ID NO: 46, PmpG, PmpF, PmpG-1, PmpE/F-2, and RplF, and an excipient.

In accordance with another aspect, the compositions according to various aspects of the invention may further comprise a MOMP polypeptide, or a fragment or portion thereof. The fragment or portion thereof may comprise SEQ ID NO: 44 or SEQ ID NO: 47.

In accordance with another aspect of the invention, there is provided a composition comprising one or more than one of PmpG, PmpF, PmpG-1, PmpE/F-2 and MOMP of C. trachomatis, and dimethyldioctadecylammonium bromide and trehalose 6,6′-dibehenate (DDA/TDB).

In accordance with another aspect of the invention, there is provided a method of identifying an antigenic epitope of a pathogen, the epitope capable of eliciting a protective immune response, the method comprising isolating antigen presenting cells from a naive subject; incubating the dendritic cells with an intracellular pathogen; isolating MHC:antigen complexes from the dendritic cells; eluting antigen from the MHC:antigen complexes, and; determining the amino acid composition of the antigenic peptide.

The antigen presenting cells may be dendritic cells.

The amino acid composition of the peptide may be determined using mass spectrometry.

In another aspect of the invention, a method is provided to elicit an immune response against C. trachomatis in mammals. The method comprises the administration of a therapeutically effective amount of a composition comprising C. trachomatis antigenic proteins.

In another aspect of the invention, the composition further comprises a carrier to improve the immunological response in a mammal. In some aspects of the invention, the carrier may comprise a liposomal delivery vehicle.

In other aspect of the invention, the composition comprises one or more recombinant antigens from C. trachomatis selected from the group of PmpG, PmpE/F and RplF including fragments and analogs thereof. The recombinant antigens may be one, or more than one, of SEQ ID NO: 42, SEQ ID NO: 43 and SEQ ID NO: 44.

The DDA/TDB adjuvant performed superior to CpG-ODN and AbISCO when combined with one or more than one of the polypeptides according to SEQ ID NO: 42-47, and also demonstrated superior IL-17 production before and after challenge. Test subjects treated with compositions comprising the DDA/TDA adjuvant also demonstrated the highest frequency of double-positive IFN-γ and IL-17 CD4+ T cells whereas CpG group or PBS controls demonstrated low to nil double-positive IFN-γ and IL-17 CD4+ T cells. These results indicate that IL-17 may have a co-operative role with IFN-γ in vaccine-primed protective immunity against Chlamydia.

The examples provided herein demonstrate that a Chlamydia vaccine based on recombinant C. muridarum proteins (PmpG-1, PmpE/F-2 and MOMP) or fragments thereof and formulated with a liposome adjuvant DDA/TDB is protective against vaginal challenge with C. muridarum. This protection correlates with strong IFN-γ, TNF-α and IL-17 responses characterized by the high frequency of IFN-γ/TNF-α double positive CD4+ T cells and IFN-γ/IL-17 double positive CD4+ T cells.

This summary of the invention does not necessarily describe all features of the invention. Other aspects, features and advantages of the present invention will become apparent to those of ordinary skill in the art upon review of the following description of specific embodiments of the invention.

### BRIEF DESCRIPTION OF THE DRAWINGS

These and other features of the invention will become more apparent from the following description in which reference is made to the appended drawings wherein:

FIG. 1 shows an exemplary schematic of vaccine development.

FIG. 2 shows the results of an ELISA assay to quantify interferon (IFN)-gamma production by CD4 T cells following exposure to dendritic cells that have been pulsed with a C. muridarum peptide; PmpG=Polymorphic membrane protein G (SEQ ID NO:13—ASPIYVDPAAAGGQPPA), PmpF=Polymorphic membrane protein F (SEQ ID NO:16—AFHLFASPAANYIHTG), L6=Ribosomal protein L/Rplf (SEQ ID NO:10—GNEVFVSPAAHIIDRPG), ACP=3-oxoacyl-(acyl carrier protein) reductase (SEQ ID NO: 11—SPGQTNYAAAKAGIIGFS), Aasf=Anti-anti-sigma factor (SEQ ID NO: 12—KLDGVSSPAVQESISE), TC0420=Hypothetical protein (SEQ ID NO: 14—DLNVTGPKIQTDVD), G3D=Glyceraldehyde 3-phosphate dehydrogenase (SEQ ID NO:17—MTTVHAATATQSVVD), Clp=ATP-dependent Clp protease proteolytic subunit (SEQ ID NO: 15—IGQEITEPLANTVIA). As controls, T cells were also cultured alone (T alone) or with dendritic cells without the addition of any peptide antigen (DC). Black bars indicate that T cells were isolated from mice that had recovered from Chlamydia infection. White bars indicate that the T cells were isolated from naive mice.

FIG. 3 shows the resistance to Chlamydia muridarum infection in mice following the adoptive transfer of dendritic cells that have been pulsed with Chlamydia peptides. LPS-treated dendritic cells were either left untreated (DC alone, black squares) or were pulsed with the eight Chlamydia muridarum MHC class II peptides (SEQ ID NOs: 10-17) (DC+ peptide, black diamonds). The dendritic cells were adoptively transferred to naïve C57BL/6 mice that were subsequently challenged intranasally with 2000 Inclusion Forming Units (IFU) of C. muridarum. The results depict the percentage body weight loss of the mice following infection.

FIG. 4 shows the results of an ELISA assay to quantify interferon (IFN)-gamma production by splenocytes recovered from mice infected with C. muridarum. Mice were infected with intranasally with 1000 IFU live C. muridarum. One month later, the splenocytes from recovered mice were harvested and stimulated with in vitro for 20 h with 2 μg/ml individual peptide corresponding to SEQ ID NO: 10-17 (white bars) or a pool of peptides corresponding to SEQ ID NO: 10-17 (pool, white bar), 1 μg/ml individual polypeptides corresponding to Ribosomal protein L6 (RplF), 3_oxoacyl_(acyl carrier protein) reductase (FabG), Anti sigma factor (Aasf), Polymorphic membrane protein G (PmpG-1), Hypothetical protein TC0420, ATP_dependent Clp_protease_proteolytic subunit (Clp), Polymorphic membrane protein F (PmpE/F) (hatched bars) or a pool of proteins (RplF, FabG, Aasf, PmpG-1, TC0420, Clp and PmpE/F). One irrelevant OVA peptide (Ctr_{neg} white bar) and GST protein (Ctr_{neg} hatched bar) were used as peptide and protein negative controls, respectively. Heat killed EB (HK-EB) was used as a positive control. MOMP protein stimulation was also set up as a reference. The results represent the average of duplicate wells and are expressed as the means±SEM of Chlamydia muridarum antigen-induced IFN-γ secreting cells per 10⁶ splenocytes for groups of six mice.

FIG. 5 shows the results of an ELISA assay to quantify interferon (IFN)-gamma production by splenocytes recovered from mice following the adoptive transfer of DCs transfected with Chlamydia muridarum polypeptides. Mice were vaccinated three times with DCs transfected with Chlamydia muridarum polypeptide PmpG-1₂₅₋₅₀₀ (PmpG-1-DC), RplF (RplF-DC), PmpE/F-2₂₅₋₅₇₅ (PmpE/F-2-DC) or MOMP (MOMP-DC) and matured overnight with LPS. DCs pulsed with live C. muridarum (EB-DC) or GST protein (GST-DC) was used as positive and negative controls, respectively. Two weeks after the last immunization, the splenocytes of each group were harvested for IFN-gamma ELISPOT assay. The results are expressed as the means±SEM of Chlamydia antigen-induced IFN-gamma secreting cells per 10⁶ splenocytes for groups of six mice.

FIG. 6 shows the resistance to Chlamydia pulmonary infection in mice following the adoptive transfer of DCs transfected with Chlamydia proteins. Mice were adoptively transferred with DCs that were transfected with either the PmpG-1₂₅₋₅₀₀ (PmpG-1-DC), RplF (RplF-DC), PmpE/F-2₂₅₋₅₇₅ (PmpE/F-2-DC), MOMP protein (MOMP-DC) or the GST protein (GST-DC). DCs pulsed with live C. muridarum (EB-DC) was used as a positive control. Two weeks after the last immunization, mice were challenged intranasally with 2000 IFU live C. muridarum. (A) Weight loss was monitored each or every two days after challenge. (B) Ten days after intranasal challenge, the lungs were collected and bacterial titers were measured on HeLa 229 cells. *, p<0.05; **, p<0.01 as compared to the GST-DC group.

FIG. 7 shows the resistance to Chlamydia genital tract infection following adoptive transfer of DCs transfected with Chlamydia proteins. Mice were adoptively transferred with DCs that were transfected with either the PmpG-1₂₅₋₅₀₀ protein (PmpG-1-DC), the RplF protein (RplF-DC), the PmpE/F-2₂₅₋₅₇₅ protein (PmpE/F-2-DC), the MOMP protein (MOMP-DC) or the GST protein (GST-DC). DCs pulsed with live C. muridarum (EB-DC) was used as a positive control. One week after the final immunization, the mice from each group were injected with Depo-Provera. One week after Depo-Provera treatment, the mice were infected intravaginally with 1500 IFU live C. muridarum. Cervicovaginal washes were taken at day 6 after infection and bacterial titer were measured on HeLa 229 cells. *, p<0.05; **, p<0.01; ***, p<0.001 as compared to the GST-DC group.

FIG. 8 Resistance to Chlamydia genital tract infection following subcutaneous vaccination with PmpG, PmpF, or MOMP protein or their combination formulated with adjuvant DDA/TDB. C57BL/6 mice were vaccinated three times with a 2-week interval with PBS, DDA/TDB alone as negative controls and live Chlamydia EB as positive control. G+F+M+DDA/TDB-PmpG, PmpF and MOMP combined with DDA/TDB. One week after the final immunization, the mice from each group were injected with Depo-Provera. One week after Depo-Provera treatment, the mice were infected intravaginally with 1500 IFU live C. muridarum. Cervicovaginal washes were taken at day 6 and day 13 after infection and bacterial titer were measured on HeLa 229 cells. The data shown above is at day 13. *, p<0.05; **, p<0.01; ***, p<0.001 vs. adjuvant alone group.

FIG. 9 shows vaccine-elicited protection against Chlamydia genital tract infection. Mice were intravaginally challenged with 1500 IFU live C. muridarum after immunization with a variety of vaccine formulation. Cervicovaginal washes were taken at selected dates after infection and bacterial titers were measured on HeLa 229 cells. *, p<0.05; **, p<0.01; ***, p<0.001 vs. adjuvant alone group. (a) Failure to induce protection after vaccination of PmpG-1 or MOMP protein formulated with CpG ODN. (b) and (c) Resistance to Chlamydia infection in C57 mice immunized with PmpG-1, PmpE/F-2, MOMP protein or their combination formulated with adjuvant AbISCO-100 or DDA/TDB. Cervicovaginal washes were taken at day 6 (b) and day 13 (c) after infection. (d) Resistance to Chlamydia infection in BALB/c mice (n=8) immunized with the combination of PmpG-1, PmpE/F-2, MOMP protein formulated with adjuvant DDA/TDB.

FIG. 10 shows Chlamydia antigen-specific cytokine response after immunization with PmpG-1 protein formulated with DDA/TDB, AbISCO or CpG adjuvants. Two weeks after the final immunization, mouse splenocytes from different vaccine groups were harvested and stimulated with 1 μg/ml PmpG-1 protein or 5×10₅ inclusion-forming units (IFU)/ml HK-EB. DDA/TDB alone, AbISCO alone or CpG alone adjuvants was set up as negative controls. The results represent the average of duplicate wells and are expressed as the means±SEM for groups of six mice. (a) IFN-γ responses to PmpG-1 and HK-EB detected by ELISPOT assay. (b) IL-17 responses to PmpG-1 and HK-EB detected by ELISPOT assay. (c) TNF-α response to PmpG-1 and HK-EB detected by ELISA.

FIG. 11 shows functional characterization of distinct populations of Chlamydia antigen-specific cytokine responses after immunization. Splenocytes from different vaccine groups were analyzed by multiparameter flow cytometry. Three or four mice were in each group. Shown is the representative of two experiments. (a) The staining panel and gating strategy used to identify IFN-γ, TNF-α and IL-17 producing CD4+ T cells in the splenocytes from a representative mouse immunized with PmpG+DDA/TDB. (b) Comparison of the quality of CD4+ IFN-γ/TNF-α responses to PmpG-1 protein (b-1) or HK-EB (b-2) in different vaccine groups. (c) Comparison of the quality of CD4+ IFN-γ/IL-17 responses to PmpG-1 protein (c-1) or HK-EB (c-2) in different vaccine groups.

FIG. 12 shows the magnitude and quality of Chlamydia antigen-specific cytokine responses in spleen and draining lymph node after challenge. Splenocytes and draining lymph node (iliac lymph node) from different vaccine groups were analyzed by multiparameter flow cytometry as described in Methods and Materials. Four mice were studied in each group. (a) The total frequency of IFN-γ, TNF-α, or IL-17 producing CD4+ T cell in spleens. (b) The total frequency of IFN-γ, TNF-α or IL-17 producing CD4+ T cell in iliac lymph node. (c) Comparison of the quality of CD4+ IFN-γ/TNF-α responses to PmpG-1 protein in spleen (c-1) and in iliac lymph node (c-2) from different vaccine groups. (d) Comparison of the quality of CD4+ IFN-γ/IL-17 responses to PmpG-1 protein in spleen (d-1) and in iliac lymph node (d-2) from different vaccine groups.

FIG. 13 shows Human Chlamydia trachomatis antigen-specific IFN-gamma response in mice after immunization with a cocktail of C. trachomatis serovar D proteins PmpG (SEQ ID NO: 42), PmpF (SEQ ID NO: 43) and MOMP (SEQ ID NO: 44) formulated with DDA/TDB adjuvant detected by ELISPOT assay. C57 BL/6 mice were immunized three times subcutaneously in the base of tail at 2-week intervals. Two weeks after the final immunization, splenocytes were harvested and stimulated with 1 microgram/ml C. trachomatis serovar D protein PmpG, PmpF, MOMP or 5×10⁵ inclusion-forming units (IFU)/ml heat-killed EB respectively. DDA/TDB alone adjuvant was set up as a negative control. The results represent the average of duplicate wells and are expressed as means±SEM for groups of six mice.

FIG. 14 shows polypeptide sequences according to SEQ ID NO: 42-47.

### DETAILED DESCRIPTION

The present invention relates to immunology, and immunostimulatory agents. More specifically, the present invention relates to compositions comprising Chlamydia antigens; the compositions may be useful for inducing an immune response to a Chlamydia spp.

In the description that follows, a number of terms are used extensively, the following definitions are provided to facilitate understanding of various aspects of the invention. Use of examples in the specification, including examples of terms, is for illustrative purposes only and is not intended to limit the scope and meaning of the embodiments of the invention herein.

Any terms not directly defined herein shall be understood to have the meanings commonly associated with them as understood within the art of the invention. Certain terms are discussed below, or elsewhere in the specification, to provide additional guidance to the practitioner in describing the devices, methods and the like of embodiments of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples in the specification, including examples of terms, is for illustrative purposes only and odes not limit the scope and meaning of the embodiments of the invention herein.

The present invention relates to compositions for inducing an immune response to a Chlamydia species in a subject. The compositions comprise one or more than one polypeptides of Chlamydia trachomatis or Chlamydia muridarum, or C. trachomatis and C. muridarum.

Chlamydia research is aided by a recognized murine model of infection that has been standardized (Brunham et al 2005. Nature Reviews Immunology 5:149-161; Taylor-Robinson and Tuffrey 1987. Infection and Immunity 24(2) 169-173; Pal et al 1998. Journal of Medical Microbiology 47(7) 599-605).

C. muridarum and C. trachomatis are highly orthologous pathogenic microbes having co-evolved with their host species. Of the approximately 1,000 genes that each organism has, all but six are shared between the two genomes. Differences in gene content between the two genomes are principally located at the replication termination region or plasticity zone. Within this region are found species specific genes that relate to host specific immune evasion mechanisms. Genes are found in C. trachomatis which encode tryptophan synthetase thereby allowing C. trachomatis to partially escape IFN-γ induced IDO-mediated tryptophan depletion in human cells. Mouse epithelial cells lack IDO and instead IFN-γ disrupts vesicular trafficking of sphingomyelin to the inclusion. C. muridarum in its genome has several genes which encode an intracellular toxin that disrupts vesicular trafficking thereby enabling partial escape from IFN-γ inhibition in murine cells.

Extraordinary gene conservation is shared between two microbial genomes. Without wishing to be bound by theory, this high degree of genome similarity may be due to the fact that as an intracellular pathogen Chlamydiae rarely undergoes lateral gene transfer events. Most genome differences result from accumulated point mutations and gene duplication. For genes shared between the two Chlamydia species, encoded proteins differ in sequence on average about 20% reflecting the extended period of time the two species have been evolutionarily separated.

In part, because the two genomes are so highly orthologous, immune responses to infection are very similar between the two host species. Because C. muridarum, like human strains, is indifferent to innate interferon gamma defenses in its natural host, clearance in the murine model is dependent on adaptive immunity, and therefore C. muridarum can serve as a robust animal model for studying cellular immunity and vaccine development. In both mice and humans CD4 T cells are particularly important to clearance of infection. Antibodies to surface macromolecules may synergise with CD4 Th1 mediated immunity in preventing reinfection. CD4 Th2 and CD4 Th17 responses in the absence of Th1 responses correlate with tissue pathology and persistent infection.

Thus, the mouse model of C. muridarum infection may be useful to elucidate the immunobiology of T cell responses and guide the design of a molecular vaccine to prevent human C. trachomatis infection.

Various Chlamydia spp have had the genome sequence determined, and the sequences of the expressed polypeptides determined. The genome sequence of C. trachomatis is described in Stephens, R. S. et al., 1998 (Genome sequence of an obligate intracellular pathogen of humans: Chlamydia trachomatis. Science 282 (5389): 754-759), the contents of which are incorporated herein by reference. Examples of expressed polypeptides of C. trachomatis that may be included in compositions according to various embodiments described herein include amino acid permease (gi:3328837), Ribosomal protein L6 (RplF, gi:3328951), 3-oxoacyl-(acyl carrier protein) reductase (FabG, gi:15604958), Anti sigma factor (Aasf, gi:15605151), Polymorphic membrane protein G (PmpG, gi:3329346), Hypothetical protein (TC0420, gi:15604862), ATP dependent Clp protease (Clp1, gi:15605439), Polymorphic membrane protein F (PmpF, gi:3329345), Glyceraldehyde 3-phosphate dehydrogenase (Gap, gi:15605234) and major outer membrane protein 1 (MOMP) (gi:3329133), or fragments or portions thereof. Examples of fragments or portions of the above-referenced polypeptides include amino acids 25-512 of PmpG (PmpG₂₅₋₅₁₂) (SEQ ID NO: 42), amino acids 26-585 of PmpF (PmpF₂₆₋₅₈₅) (SEQ ID NO: 43), and amino acids 22-393 of MOMP (SEQ ID NO: 44).

The genome sequence of C. muridarum is described in Read, T., et al., 2000 (Genome sequences of Chlamydia trachomatis MoPn and Chlamydia pneumoniae AR39 Nucleic Acids Res. 28 (6): 1397-1406), the contents of which are incorporated herein by reference. Examples of expressed polypeptides of C. muridarum that may be included in compositions according to various embodiments described herein, or employed in various experimental examples described herein include amino acid permease (gi:15835268), Ribosomal protein L6 (RplF, gi: 15835415), 3_oxoacyl_(acyl carrier protein) reductase (FabG, gi:15835126), Anti sigma factor (Aasf, gi:15835322), Polymorphic membrane protein G (PmpG or PmpG-1, gi:15834883), Hypothetical protein TC0420(gi:15835038), ATP_dependent Clp protease_proteolytic subunit (Clp, gi:15834704), Polymorphic membrane protein F (PmpF or PmpE/F, gi:15834882), Glyceraldehyde 3_phosphate dehydrogenase (Gap, gi:15835406) and major outer membrane protein 1 (MOMP, gi7190091), or fragments or portions thereof. Examples of fragments or portions of the above-referenced polypeptides include amino acids 25-500 of PmpG-1 (PmpG-1₂₅₋₅₀₀) (SEQ ID NO: 45), amino acids 25-575 of PmpE/F-2 (PmpE/F-2₂₅₋₅₇₅) (SEQ ID NO: 46), and amino acids 23-387 end of MOMP (SEQ ID NO: 47).

The nucleotide and amino acid sequences of MOMP are also described in, for example, U.S. Pat. No. 6,838,085 and U.S. Pat. No. 6,344,302, the contents of which are incorporated herein by reference.

In some embodiments of the invention, the one, or more than one, polypeptide may be selected from the group consisting of SEQ ID NO: 10, SEQ ID NO: 13, SEQ ID NO: 16, SEQ ID NO:17, SEQ ID NO: 20, SEQ ID NO: 21, SEQ ID NO: 22, SEQ ID NO: 23, SEQ ID NO: 27, SEQ ID NO: 28, SEQ ID NO: 31, SEQ ID NO: 42, SEQ ID NO: 43, SEQ ID NO: 44, SEQ ID NO: 45, SEQ ID NO: 46, SEQ ID NO: 47 and RplF, and an excipient.

The one or more than one polypeptides may be from Chlamydia trachomatis, or C. muridarum.

A fragment or portion of a protein, fusion protein or polypeptide includes a peptide or polypeptide comprising a subset of the amino acid complement of a particular protein or polypeptide. The fragment may, for example, comprise an antigenic region or a region comprising a functional domain of the protein or polypeptide. The fragment may also comprise a region or domain common to proteins of the same general family, or the fragment may include sufficient amino acid sequence to specifically identify the full-length protein from which it is derived. In some embodiments, the fragment may specifically exclude signal peptides for translocation to organelles or membranes of the cell. In some embodiments, the fragment may comprise a region or domain found on the external surface of the cell (e.g. an outer membrane protein or portion thereof) when the polypeptide is expressed in the organism or cell.

For example, a fragment or portion may comprise from about 20% to about 100%, of the length of the full length of the protein, or any amount therebetween. For example, from about 20% to about 100%, 30% to about 100%, 40% to about 100%, 50% to about 100%, 60% to about 100%, from about 70% to about 100%, from about 80% to about 100%, from about 90% to about 100%, from about 95% to about 100%, of the length of the full length of the protein, or any amount therebetween. Alternately, a fragment or portion may be from about 50 to about 500 amino acids, or any amount therebetween. For example, a fragment may be from 50 to about 500 amino acids, or any amount therebetween, from about 75 to about 500 amino acids or any amount therebetween, from about 100 to about 500 amino acids or any amount therebetween, from about 125 to about 500 amino acids or any amount therebetween, from about 150 to about 500 amino acids, or any amount therebetween, from about 200 to about 500 amino acids, or any amount therebetween, from about 250 to about 500 amino acids, or any amount therebetween, from about 300 to about 500 or any amount therebetween, from about 350 to about 500 amino acids, or any amount therebetween, from about 400 to about 500 or any amount therebetween, from about 450 to about 500 or any amount therebetween, depending upon the HA, and provided that the fragment can form a VLP when expressed. For example, about 5, 10, 20, 30, 40 or 50 amino acids, or any amount therebetween may be removed from the C terminus, the N terminus or both the N and C terminus.

Numbering of amino acids in any given sequence are relative to the particular sequence, however one of skill can readily determine the ‘equivalency’ of a particular amino acid in a sequence based on structure and/or sequence. For example, if 6 N terminal amino acids were removed when constructing a clone for crystallography, this would change the specific numerical identity of the amino acid (e.g. relative to the full length of the protein), but would not alter the relative position of the amino acid in the structure.

The present invention further provides for a method of inducing or eliciting an immune response against C. trachomatis or C. muridarum in a subject, comprising administration of a composition comprising one or more C. trachomatis, or C. muridarum, or C. trachomatis and C. muridarum polypeptides, and an excipient. The composition may further comprise an adjuvant, a delivery agent, or an adjuvant and a delivery agent.

Antigen presenting cells (APCs) such as dendritic cells (DCs) take up polypeptides and present epitopes of such polypeptides within the context of the DC MHC I and II complexes to other immune cells including CD4+ and CD8+ cells. An ‘MHC complex’ or ‘MHC receptor’ is a cell-surface receptor encoded by the major histocompatibility complex of a subject, with a role in antigen presentation for the immune system. MHC proteins may be found on several cell types, including antigen presenting cells (APCs) such as macrophages or dendritic cells (DCs), or other cells found in a mammal. Epitopes associated with MHC Class I may range from about 8-11 amino acids in length, while epitopes associated MHC Class II may be longer, ranging from about 9-25 amino acids in length.

The term “epitope” refers to an arrangement of amino acids in a protein or modifications thereon (for example glycosylation). The amino acids may be arranged in a linear fashion, such as a primary sequence of a protein, or may be a secondary or tertiary arrangement of amino acids in close proximity once a protein is partially or fully configured. Epitopes may be specifically bound by an antibody, antibody fragment, peptide, peptidomimetic or the like, or may be specifically bound by a ligand or held within an MHC I or MHC II complex. An epitope may have a range of sizes—for example a linear epitope may be as small as two amino acids, or may be larger, from about 3 amino acids to about 20 amino acids. In some embodiments, an epitope may be from about 5 amino acids to about 10 or about 15 amino acids in length. An epitope of secondary or tertiary arrangements of amino acids may encompass as few as two amino acids, or may be larger, from about 3 amino acids to about 20 amino acids. In some embodiments, a secondary or tertiary epitope may be from about 5 amino acids to about 10 or about 15 amino acids in proximity to some or others within the epitope.

An “immune response” generally refers to a response of the adaptive immune system. The adaptive immune system generally comprises a humoral response, and a cell-mediated response. The humoral response is the aspect of immunity that is mediated by secreted antibodies, produced in the cells of the B lymphocyte lineage (B cell). Secreted antibodies bind to antigens on the surfaces of invading microbes (such as viruses or bacteria), which flags them for destruction. Humoral immunity is used generally to refer to antibody production and the processes that accompany it, as well as the effector functions of antibodies, including Th2 cell activation and cytokine production, memory cell generation, opsonin promotion of phagocytosis, pathogen elimination and the like. The terms “modulate” or “modulation” or the like refer to an increase or decrease in a particular response or parameter, as determined by any of several assays generally known or used, some of which are exemplified herein. The cellular processes involved in stimulation of B-cells and T-cells are well described in the art, in various texts and references. See, for example, Roitt's Essential Immunology. I M Roitt, P J Delves. Oxford, Blackwell Science Publishers 2001

A cell-mediated response is an immune response that does not involve antibodies but rather involves the activation of macrophages, natural killer cells (NK), antigen-specific cytotoxic T-lymphocytes, and the release of various cytokines in response to an antigen. Cell-mediated immunity is used generally to refer to some Th cell activation, Tc cell activation and T-cell mediated responses. Cell mediated immunity is of particular importance in responding to viral infections.

For example, the induction of antigen specific CD8 positive T lymphocytes may be measured using an ELISPOT assay; stimulation of CD4 positive T-lymphocytes may be measured using a proliferation assay. Anti-influenza antibody titres may be quantified using an ELISA assay; isotypes of antigen-specific or cross reactive antibodies may also be measured using anti-isotype antibodies (e.g. anti-IgG, IgA, IgE or IgM). Methods and techniques for performing such assays are well-known in the art.

Cytokine presence or levels may also be quantified. For example a T-helper cell response (Th1/Th2) will be characterized by the measurement of IFN-γ and IL-4 secreting cells using by ELISA (e.g. BD Biosciences OptEIA kits). Peripheral blood mononuclear cells (PBMC) or splenocytes obtained from a subject may be cultured, and the supernatant analyzed. T lymphocytes may also be quantified by fluorescence-activated cell sorting (FACS), using marker specific fluorescent labels and methods as are known in the art.

In one example of stimulation of an adaptive immune response, a dendritic cell may engulf an exogenous pathogen, or macromolecules comprising pathogen antigenic epitopes. The phagocytosed pathogen or macromolecules are processed by the cell, and smaller fragments (antigens) are displayed on the outer surface of the cell in the context of an MHC molecule. This MHC-antigen complex may subsequently be recognized by B- or T-cells. The recognition of the MHC-antigen complex by a B- or T-cell initiates a cascade of events, including clonal expansion of the particular lymphocyte, with an outcome being a specific, pathogen-directed immune response that kills cells infected with the pathogen. Aspects of the various events involved in the cascading immune response are known in the art, as may be found in Roitt, supra.

The term “subject” or “patient” generally refers to mammals and other animals including humans and other primates, companion animals, zoo, and farm animals, including, but not limited to, cats, dogs, rodents, rats, mice, hamsters, rabbits, horses, cows, sheep, pigs, elk or other ungulates, goats, poultry, etc. The subject may have been previously assessed or diagnosed using other methods, such as those described herein or those in current clinical practice, or may be selected as part of a general population (a control subject).

In some embodiments, the present invention also provides for a composition for inducing an immune response in a subject. Compositions according to various embodiments of the invention may be used as a vaccine, or in the preparation of a vaccine.

The term ‘vaccine’ refers to an antigenic preparation that may be used to establish an immune response to a polypeptide, protein, glycoprotein, lipoprotein or other macromolecule. The immune response may be highly specific, for example directed to a single epitope comprising a portion of the macromolecule, or may be directed to several epitopes, one or more of which may comprise a portion of the macromolecule. Vaccines are frequently developed so as to direct the immune response to a pathogen. The immune response may be prophylactic, with the goal of preventing or ameliorating the effect of a future infection by a particular pathogen, or may be therapeutic, and administered with the goal of supplementing or stimulating a stronger immune response to one or more epitopes.

Several types of vaccines are known in the art. An inactivated vaccine is a vaccine comprising a previously killed pathogenic microorganism. Examples of killed vaccines include those for some influenza strains and hepatitis A live/attenuated vaccine comprises a non-killed pathogen that has been manipulated genetically, or grown under particular conditions, so that the virulence of the pathogen is reduced. Examples of live/attenuated vaccines include those for measles, mumps or rubella. A subunit vaccine is a vaccine comprising a fragment of the pathogenic microorganism. The fragment may include particular surface proteins or markers, or portions of surface proteins or markers, or other polypeptides that may be unique to the pathogen. Examples of subunit vaccines include vaccines include those described herein. Adjuvants, excipients, other additives for inclusion in a composition for use in a vaccine and methods of preparing such compositions will be known to those of skill in the art.

The terms ‘peptide’, ‘polypeptide’ and protein’ may be used interchangeably, and refer to a macromolecule comprised of at least two amino acid residues covalently linked by peptide bonds or modified peptide bonds, for example peptide isosteres (modified peptide bonds) that may provide additional desired properties to the peptide, such as increased half-life. A peptide may comprise at least two amino acids. The amino acids comprising a peptide or protein described herein may also be modified either by natural processes, such as posttranslational processing, or by chemical modification techniques which are well known in the art. Modifications can occur anywhere in a peptide, including the peptide backbone, the amino acid side-chains and the amino or carboxyl termini. It is understood that the same type of modification may be present in the same or varying degrees at several sites in a given peptide.

Examples of modifications to peptides may include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for example, Wold F, Posttranslational Protein Modifications: Perspectives and Prospects, pgs. 1-12 in Posttranslational Covalent Modification of Proteins, B. C. Johnson, ed., Academic Press, New York, 1983; Seifter et al., Analysis for protein modifications and nonprotein cofactors, Meth. Enzymol. (1990) 182: 626-646 and Rattan et al. (1992), Protein Synthesis: Posttranslational Modifications and Aging,” Ann NY Acad Sci 663: 48-62.

A substantially similar sequence is an amino acid sequence that differs from a reference sequence only by one or more conservative substitutions. Such a sequence may, for example, be functionally homologous to another substantially similar sequence. It will be appreciated by a person of skill in the art the aspects of the individual amino acids in a peptide of the invention that may be substituted.

Amino acid sequence similarity or identity may be computed by using the BLASTP and TBLASTN programs which employ the BLAST (basic local alignment search tool) 2.0 algorithm. Techniques for computing amino acid sequence similarity or identity are well known to those skilled in the art, and the use of the BLAST algorithm is described in ALTSCHUL et al. 1990, J Mol. Biol. 215: 403-410 and ALTSCHUL et al. (1997), Nucleic Acids Res. 25: 3389-3402.

Standard reference works setting forth the general principles of peptide synthesis technology and methods known to those of skill in the art include, for example: Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994).

A protein or polypeptide, or fragment or portion of a protein or polypeptide is specifically identified when its sequence may be differentiated from others found in the same phylogenetic Species, Genus, Family or Order. Such differentiation may be identified by comparison of sequences. Comparisons of a sequence or sequences may be done using a BLAST algorithm (Altschul et al. 1009. J. Mol. Biol 215:403-410). A BLAST search allows for comparison of a query sequence with a specific sequence or group of sequences, or with a larger library or database (e.g. GenBank or GenPept) of sequences, and identify not only sequences that exhibit 100% identity, but also those with lesser degrees of identity. For proteins with multiple isoforms, an isoform may be specifically identified when it is differentiated from other isoforms from the same or a different species, by specific detection of a structure, sequence or motif that is present on one isoform and is absent, or not detectable on one or more other isoforms.

It will be appreciated by a person of skill in the art that any numerical designations of amino acids within a sequence are relative to the specific sequence. Also, the same positions may be assigned different numerical designations depending on the way in which the sequence is numbered and the sequence chosen. Furthermore, sequence variations such as insertions or deletions, may change the relative position and subsequently the numerical designations of particular amino acids at and around a site or element of secondary or tertiary structure.

Nomenclature used to describe the peptides of the present invention follows the conventional practice where the amino group is presented to the left and the carboxy group to the right of each amino acid residue. In the sequences representing selected specific embodiments of the present invention, the amino- and carboxy-terminal groups, although not specifically shown, will be understood to be in the form they would assume at physiologic pH values, unless otherwise specified. In the amino acid structure formulae, each residue may be generally represented by a one-letter or three-letter designation, corresponding to the trivial name of the amino acid, such as is known in the art

Amino acids comprising the peptides described herein will be understood to be in the L- or D-configuration. In peptides and peptidomimetics of the present invention, D-amino acids may be substitutable for L-amino acids.

A peptidomimetic is a compound comprising non-peptidic structural elements that mimics the biological action of a parent peptide. A peptidomimetic may not have classical peptide characteristics such as an enzymatically scissile peptidic bond. A parent peptide may initially be identified as a binding sequence or phosphorylation site on a protein of interest, or may be a naturally occurring peptide, for example a peptide hormone. Assays to identify peptidomimetics may include a parent peptide as a positive control for comparison purposes, when screening a library, such as a peptidomimetic library. A peptidomimetic library is a library of compounds that may have biological activity similar to that of a parent peptide.

Amino acids may be substitutable, based on one or more similarities in the R-group or side-chain constituents, for example, hydropathic index, polarity, size, charge, electrophilic character, hydrophobicity and the like.

Peptides according to one embodiment of the invention may include peptides comprising the amino acid sequences according to SEQ ID NOs: 10-17 and 19-32. Other peptides according to other embodiments of the invention may include peptides having a substantially similar sequence to that of SEQ ID NOs: 10-17 and 19-32. Polypeptides according to some embodiments of the invention may include polypeptides having a substantially similar sequence to that of amino acid permease, RplF, FabG, Aasf, PmpG-1, TC0420, Clp1, PmpE/F-2, Gap, or MOMP, or SEQ ID NO: 42, SEQ ID NO: 43, SEQ ID NO: 44, SEQ ID NO: 45, SEQ ID NO: 46 or SEQ ID NO: 47, or fragments or portions thereof. Such peptides or proteins may be in isolation or in combination and may be linked to, or in combination with, tracer compounds, protein translocation sequences, liposomes, carbohydrate carriers, polymeric carriers or other agents or excipients as will be apparent to one of skill in the art.

It will be appreciated by a person of skill in the art that the numerical designations of the positions of amino acids within a sequence are relative to the specific sequence. Also the same positions may be assigned different numerical designations depending on the way in which the sequence is numbered and the sequence chosen. Furthermore, sequence variations such as insertions or deletions, may change the relative position and subsequently the numerical designations of particular amino acids at and around a site.

The adaptive immune response is exploited by vaccination to provide an immunological advantage to an otherwise naïve subject. A vaccine may comprise immunogens that provide specific stimulation of an adaptive immune response to a virulent pathogen to which a subject has not yet been exposed.

An immunoproteomic approach to identifying candidate T-cell antigens may avoid the introduction of bias and maintain fidelity with antigen processing in a natural infection. An epitope that is never presented in the context of an MHC molecule will not be able to interact with immune effector cells such as T-cells or B-cells. On the other hand, an epitope identified by virtue of association with an MHC molecule may be able to interact with an immune effector cell, and thus have a greater likelihood of eliciting a suitable immune response.

Identification of an MHC-associated epitope from an antigen-presenting cell may be facilitated by enrichment of a cell lysate for MHC molecules, and release of peptides from the MHC complex. Methods of enriching a cell lysates for the MHC molecule fraction are known in the art and may include immunological methods such as immunoaffinity chromatography. Methods of releasing peptides from an MHC complex are known in the art and may include mild acidification of the lysate following enrichment. See, for example, Current Protocols in Immunology J E Coligan, ed. Wiley InterScience.

Identification of MHC-associated epitopes may be further facilitated by proteomics methods suited to analysis of minute quantities of proteins or peptides. Any given antigen presenting cell (APC) such as a dendritic cell (DC) may only ‘present’ one or two peptides in the MHC complexes. Further, ex vivo culture of an APC may be limited to the scale to which the APCs may be cultured. Sufficient sensitivity to enable analysis of femtomole-range concentration of peptides or proteins may be necessary. Fourier transform mass-spectrometry may provide such sensitivity. Examples of such mass spectrometers are known, and may include a linear ion trap-orbitrap (LTQ-Orbitrap) mass spectrometer (Makarov et al 2006. J. Am Soc Mass Spectrom 17:977-82), or a linear ion trap-Fourier Transform (LTQ-FT) mass spectrometer (deSouza et al 2006. 7:R72).

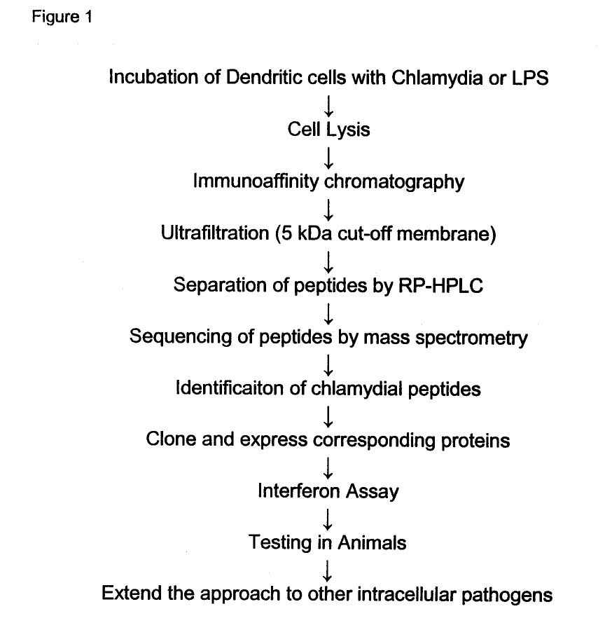

A schematic representation of an exemplary method involved in an immunoproteomics approach to identifying candidate T-cell antigens as described herein is shown in FIG. 1.

Dendritic cells are isolated from a subject and co-incubated with an intracellular pathogen, for example C. trachomatis or C. muridarum. A preparation of bacterial LPS is included as a control. Following an incubation period, for example 24-48 h, the dendritic cells are collected and lysed. Cells may be lysed by a variety of methods that preserve the MHC:antigen complex, for example sonication, lysis with mild detergents such as NP-40 or CHAPS, or with a hypotonic solution. Following cell lysis, MHC:antigen complexes may be isolated using immunogenic methods. For example, cellular debris following lysis is removed by centrifugation and the resulting supernatant comprising MHC:antigen complexes applied to an immunoaffinity column. The MHC:antigen complexes bound to the column are subsequently treated to release the antigen. For example, the column may be mildly acidified to selectively elute the antigens, leaving the MHC bound to the column. The column eluate may subsequently be concentrated by ultracentrifugation and applied to an reverse phase-HPLC, and as the antigens are eluted from the HPLC, the peptide sequence is determined by mass spectrometry.

Antigens found to associate with the MHC of dendritic cells may be identified in this manner, and such antigens may be immunogenic.

In order to further characterize a peptide or protein, nucleic acid encoding such a peptide or related proteins or fragments thereof may be cloned and expressed in a heterologous system. Methods of producing and manipulating such nucleic acids are known in the art, and are described in, for example Ausubel, et al., Current Protocols in Molecular Biology, John Wiley & Sons, New York, N.Y. (1987-2006); or Sambrook et al., Molecular Cloning: A Laboratory Manual, 2^{nd} edition, Cold Spring Harbour, N.Y. (1989). Examples of such heterologous systems are known in the art and may include the pET system, a Baculovirus expression system, a yeast expression system, a mammalian expression system or the like. Alternatively, the peptides identified by the methods disclosed herein may be synthesized by chemical means that are known in the art.

The resulting peptide(s) or protein(s) may be, for example, exposed to cells cultured from a previously inoculated animal. The exposed cells may be assessed using an interferon gamma assay. Confirmation of the immunogenicity of the recombinant peptide(s) or protein(s) may be achieved by combining the recombinant peptide(s) or protein(s) with dendritic cells and T-cells in vitro. When the protein is processed by the dendritic cells and presented to the T-cells, an immunogenic protein will cause the T-cells to produce interferon gamma. The presence of interferon gamma in the supernatant confirms the immunogenicity of the protein or combination of proteins applied to the well. Examples of interferon assays are known in the art, and are described in, for example Rey-Ladino et al 2005 Infection and Immunity 73:1568-1577; Neild et al 2003. Immunity 18:813-823. It is within the ability of one of skill in the art to make any minor modifications to adapt such assays to a particular cellular model.

In another embodiment, a candidate T cell antigen as described above may be used to inoculate a test subject, for example, an animal model of Chlamydia infection, such as a mouse. Methods of experimentally inoculating experimental animals are known in the art. For example, testing a Chlamydia spp. vaccine may involve infecting previously inoculated mice intranasally with an inoculum comprising an infectious Chlamydia strain, and assessing for development of pneumonia. An exemplary assay is described in, for example Tammiruusu et al 2007. Vaccine 25(2):283-290, or in Rey-Ladino et al 2005. Infection and Immunity 73:1568-1577. It is within the ability of one of skill in the art to make any minor modifications to adapt such an assay to a particular pathogen model.

In another example, testing a Chlamydia vaccine may involve serially inoculating female mice with a candidate T-cell antigen cloned and expressed as described above. A series of inoculations may comprise two, three or more serial inoculations. The candidate T-cell antigens may be combined with an adjuvant. About three weeks following the last inoculation in the series, mice are treated subcutaneously with 2.5 mg Depo-Provera and one week later both naïve and immunized mice may be infected intravaginally with Chlamydia. The course of infection may be followed by monitoring the number of organisms shed at 2 to 7 day intervals for 6 weeks. The amount of organism shed may be determined by counting Chlamydia inclusion formation in Hela cells using appropriately diluted vaginal wash samples. Immunity may be measured by the reduction in the amount of organism shed in immunized mice compared to naïve mice.

In another embodiment of the invention, a combination of two, three, four or more candidate T-cell antigens may be co-inoculated in an experimental animal, or exposed to cells from an inoculated animal.

In another example, peptides comprising one, or more than one, of SEQ ID NO: 10, SEQ ID NO: 13, SEQ ID NO: 16, SEQ ID NO:17, SEQ ID NO: 20, SEQ ID NO: 21, SEQ ID NO: 22, SEQ ID NO: 23, SEQ ID NO: 27, SEQ ID NO: 28, SEQ ID NO: 31, PmpG-1, PmpE/F-2, SEQ ID NO: 42, SEQ ID NO: 43, SEQ ID NO: 45, SEQ ID NO: 46, and RplF may be used in a pharmaceutical preparation for inducing an immune response to one or more than one Chlamydia epitopes. The pharmaceutical preparation may be useful as a vaccine.

The pharmaceutical preparation may further comprise a polypeptide corresponding to one or more of SEQ ID NO: 44 or SEQ ID NO: 47.

In another embodiment of the invention, a peptide may be used in the preparation of a medicament such as a vaccine composition, for the prevention or treatment of a Chlamydia infection. The peptide, or medicament or vaccine composition comprising the peptide, may be used for the prevention or treatment of a Chlamydia infection in a subject having, or suspected of having such a disease or disorder.

An “effective amount” of a peptide or polypeptide as used herein refers to the amount of peptide or polypeptide in the pharmaceutical composition to induce an immune response to a Chlamydia epitope in a subject. The effective amount may be calculated on a mass/mass basis (e.g. micrograms or milligrams per kilogram of subject), or may be calculated on a mass/volume basis (e.g. concentration, micrograms or milligrams per milliliter). Using a mass/volume unit, one or more peptides or polypeptides may be present at an amount from about 0.1 ug/ml to about 20 mg/ml, or any amount therebetween, for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/ml, or any amount therebetween; or from about 1 ug/ml to about 2000 ug/ml, or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, ug/ml or any amount therebetween; or from about 10 ug/ml to about 1000 ug/ml or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/ml, or any amount therebetween; or from about 30 ug/ml to about 1000 ug/ml or any amount therebetween, for example 30.0, 35.0, 40.0, 50.0 60.0, 70.0, 80.0, 90.0, 100, 120, 140, 160 180, 200, 250, 500, 750, 1000 ug/ml.

Quantities and/or concentrations may be calculated on a mass/mass basis (e.g. micrograms or milligrams per kilogram of subject), or may be calculated on a mass/volume basis (e.g. concentration, micrograms or milligrams per milliliter). Using a mass/volume unit, one or more peptides or polypeptides may be present at an amount from about 0.1 ug/ml to about 20 mg/ml, or any amount therebetween, for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/ml, or any amount therebetween; or from about 1 ug/ml to about 2000 ug/ml, or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, ug/ml or any amount therebetween; or from about 10 ug/ml to about 1000 ug/ml or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/ml, or any amount therebetween; or from about 30 ug/ml to about 1000 ug/ml or any amount therebetween, for example 30.0, 35.0, 40.0, 50.0 60.0, 70.0, 80.0, 90.0, 100, 120, 140, 160 180, 200, 250, 500, 750, 1000 ug/ml.

Compositions according to various embodiments of the invention, including therapeutic compositions, may be administered as a dose comprising an effective amount of one or more peptides or polypeptides. The dose may comprise from about 0.1 ug/kg to about 20 mg/kg (based on the mass of the subject), for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/kg, or any amount therebetween; or from about 1 ug/kg to about 2000 ug/kg or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000 ug/kg, or any amount therebetween; or from about 10 ug/kg to about 1000 ug/kg or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/kg, or any amount therebetween; or from about 30 ug/kg to about 1000 ug/kg or any amount therebetween, for example 30.0, 35.0, 40.0, 50.0 60.0, 70.0, 80.0, 90.0, 100, 120, 140, 160 180, 200, 250, 500, 750, 1000 ug/kg.

One of skill in the art will be readily able to interconvert the units as necessary, given the mass of the subject, the concentration of the composition, individual components or combinations thereof, or volume of the composition, individual components or combinations thereof, into a format suitable for the desired application.

The amount of a composition administered, where it is administered, the method of administration and the timeframe over which it is administered may all contribute to the observed effect. As an example, a composition may be administered systemically e.g. intravenous administration and have a toxic or undesirable effect, while the same composition administered subcutaneously or intranasally may not yield the same undesirable effect. In some embodiments, localized stimulation of immune cells in the lymph nodes close to the site of subcutaneous injection may be advantageous, while a systemic immune stimulation may not.

Compositions according to various embodiments of the invention may be formulated with any of a variety of physiologically or pharmaceutically acceptable excipients, frequently in an aqueous vehicle such as Water for Injection, Ringer's lactate, isotonic saline or the like. Such excipients may include, for example, salts, buffers, antioxidants, complexing agents, tonicity agents, cryoprotectants, lyoprotectants, suspending agents, emulsifying agents, antimicrobial agents, preservatives, chelating agents, binding agents, surfactants, wetting agents, anti-adherents agents, disentegrants, coatings, glidants, deflocculating agents, anti-nucleating agents, surfactants, stabilizing agents, non-aqueous vehicles such as fixed oils, polymers or encapsulants for sustained or controlled release, ointment bases, fatty acids, cream bases, emollients, emulsifiers, thickeners, preservatives, solubilizing agents, humectants, water, alcohols or the like. See, for example, Berge et al. (1977. J. Pharm Sci. 66:1-19), or Remington—The Science and Practice of Pharmacy, 21^{st} edition. Gennaro et al editors. Lippincott Williams & Wilkins Philadelphia (both of which are herein incorporated by reference).

Compositions comprising one or more peptides or polypeptides according to various embodiments of the invention may be administered by any of several routes, including, for example and without limitation, intrathecal administration, subcutaneous injection, intraperitoneal injection, intramuscular injection, intravenous injection, epidermal or transdermal administration, mucosal membrane administration, orally, nasally, rectally, topically or vaginally. See, for example, Remington—The Science and Practice of Pharmacy, 21^{st} edition. Gennaro et al editors. Lippincott Williams & Wilkins Philadelphia. Carrier formulations may be selected or modified according to the route of administration.

Compositions according to various embodiments of the invention may be applied to epithelial surfaces. Some epithelial surfaces may comprise a mucosal membrane, for example buccal, gingival, nasal, tracheal, bronchial, gastrointestinal, rectal, urethral, vaginal, cervical, uterine and the like. Some epithelial surfaces may comprise keratinized cells, for example, skin, tongue, gingival, palate or the like.

Compositions according to various embodiments of the invention may be provided in a unit dosage form, or in a bulk form suitable for formulation or dilution at the point of use.

Compositions according to various embodiments of the invention may be administered to a subject in a single-dose, or in several doses administered over time. Dosage schedules may be dependent on, for example, the subject's condition, age, gender, weight, route of administration, formulation, or general health. Dosage schedules may be calculated from measurements of adsorption, distribution, metabolism, excretion and toxicity in a subject, or may be extrapolated from measurements on an experimental animal, such as a rat or mouse, for use in a human subject. Optimization of dosage and treatment regimens are discussed in, for example, Goodman & Gilman's The Pharmacological Basis of Therapeutics 11^{th} edition. 2006. L L Brunton, editor. McGraw-Hill, New York, or Remington—The Science and Practice of Pharmacy, 21^{st} edition. Gennaro et al editors. Lippincott Williams & Wilkins Philadelphia.

Compositions for use as vaccine compositions according to various embodiments of the invention may further comprise an adjuvant and administered as described. For example, a peptide or polypeptide for use in a vaccine composition may be combined with an adjuvant, examples of adjuvants include aluminum hydroxide, alum, Alhydrogel™ (aluminum trihydrate) or other aluminum-comprising salts, virosomes, nucleic acids comprising CpG motifs, squalene, oils, MF59, QS21, various saponins, virus-like particles, monophosphoryl-lipid A (MPL)/trehalose dicorynomycolate, toll-like receptor agonists, copolymers such as polyoxypropylene and polyoxyethylene, AbISCO, montanide ISA-51 or the like. In some embodiments, the one or more peptides or polypeptides may be combined with a cationic lipid delivery agent (Dimethyldioctadecylammonium Bromide (DDA) together with a modified mycobacterial cord factor trehalose 6,6′-dibehenate (TDB). Liposomes with or without incorporated MPL further been adsorbed to alum hydroxide may also be useful, see, for example U.S. Pat. Nos. 6,093,406 and 6,793,923 B2.

In the context of the present invention, the terms “treatment,”, “treating”, “therapeutic use,” or “treatment regimen” as used herein may be used interchangeably are meant to encompass prophylactic, palliative, and therapeutic modalities of administration of the compositions of the present invention, and include any and all uses of the presently claimed compounds that remedy a disease state, condition, symptom, sign, or disorder caused by an inflammation-based pathology, infectious disease, allergic response, hyperimmune response, or other disease or disorder to be treated, or which prevents, hinders, retards, or reverses the progression of symptoms, signs, conditions, or disorders associated therewith.

Standard reference works setting forth the general principles of immunology known to those of skill in the art include, for example: Harlow and Lane, Antibodies: A Laboratory Manual, 2d Ed., Cold Spring Harbor Laboratory Press, Cold Spring Harbor, N.Y. (1999); Harlow and Lane, Using Antibodies: A Laboratory Manual. Cold Spring Harbor Laboratory Press, New York; Coligan et al. eds. Current Protocols in Immunology, John Wiley & Sons, New York, N.Y. (1992-2006); and Roitt et al., Immunology, 3d Ed., Mosby-Year Book Europe Limited, London (1993).

Design and selection of primers for PCR amplification of sequences will readily be apparent to those of skill in the art when provided with one or more nucleic acid sequences comprising the sequence to be amplified. Selection of such a sequence may entail determining the nucleotide sequence encoding a desired polypeptide, including initiation and termination signals and codons. A skilled worker, when provided with the nucleic acid sequence, or a polypeptide sequence encoded by the desired nucleic acid sequence, will be able to ascertain one or more suitable segments of the nucleic acid to be amplified, and select primers or other tools accordingly. Standard reference works setting forth the general principles of recombinant DNA technology known to those of skill in the art include, for example: Ausubel et al, Current Protocols In Molecular Biology, John Wiley & Sons, New York (1998 and Supplements to 2001); Sambrook et al, Molecular Cloning: A Laboratory Manual, 2d Ed., Cold Spring Harbor Laboratory Press, Plainview, N.Y. (1989); Kaufman et al, Eds., Handbook Of Molecular And Cellular Methods In Biology And Medicine, CRC Press, Boca Raton (1995); McPherson, Ed., Directed Mutagenesis: A Practical Approach, IRL Press, Oxford (1991)

Alternate embodiments: An alternative method to generate MHC-bound peptides for subsequent analysis using the mass spectrophotometric methods described herein includes use of an immortalized DC line from C57BL/6 mice transfected with myc and ras oncogenes that are immunologically equivalent to primary DCs, expressing high levels of MHC (Shen Z 1997 J. Immunol. 158:2723-2730). Such immortalized dendritic cells may be exposed to proteins or peptides having Chlamydial epitopes, and used in the methods described herein.

Compositions may be used as vaccine formulations and tested in nonhuman primates at a suitable facility, such as the University of Washington's Primate Centre. Groups of suitable primates, e.g. Cynomolgus macaques, may be immunized with adjuvant alone as negative control, PmpG or SEQ ID NO: 42, PmpF or SEQ ID NO: 43, MOMP or SEQ ID NO: 44, or combinations thereof with an adjuvant or PmpG or SEQ ID NO: 42, PmpF or SEQ ID NO: 43, and MOMP or SEQ ID NO: 44 pooled and combined with an adjuvant. The compositions may be administered to the subjects by injection (e.g. intramuscular injection) with an effective dose (e.g. 100 micrograms per antigen at day 0 and months 1 and 3). Following the administration schedule, at four months the subjects may be challenged intracervically with 10×1050 serovar 0 C. trachomatis and followed at weekly intervals with quantitative cultures and NAAT tests for four months or until clearing occurs. At eight months after the initial administration, the animals may be examined by laparoscopy (or ultrasound or MRI) to visually define the upper genital tract pathology. Serum and peripheral blood cells may be collected at baseline, 1, 3, and 4 though 8 months and prior to Imaging.

Articles of Manufacture

Also provided is an article of manufacture, comprising packaging material and a composition comprising one or more peptides or polypeptides as provided herein. The composition includes a physiologically or pharmaceutically acceptable excipient, and may further include an adjuvant, a delivery agent, or an adjuvant and a delivery agent, and the packaging material may include a label which indicates the active ingredients of the composition (e.g. the peptide or polypeptide, adjuvant or delivery agent as present). The label may further include an intended use of the composition, for example as a therapeutic or prophylactic composition to be used in the manner described herein.

Kits

In another embodiment, a kit for the preparation of a medicament, comprising a composition comprising one or more peptides as provided herein, along with instructions for its use is provided. The instructions may comprise a series of steps for the preparation of the medicament, the medicament being useful for inducing a therapeutic or prophylactic immune response in a subject to whom it is administered. The kit may further comprise instructions for use of the medicament in treatment for treatment, prevention or amelioration of one or more symptoms of a Chlamydia infection, and include, for example, dose concentrations, dose intervals, preferred administration methods or the like.

The present invention will be further illustrated in the following examples. However it is to be understood that these examples are for illustrative purposes only, and should not be used to limit the scope of the present invention in any manner.

Methods

Mice: Female C57BL/6 mice (8 to 10 weeks old) were purchased from Charles River Canada (Saint Constant, Canada).

Dendritic cell generation from bone marrow: Dendritic cells (DCs) were generated following the protocol described by Lutz et al. 1999 J Immunol Methods 223:77-92. Briefly, bone marrow cells were prepared from the femora and tibiae of naïve C57BL/6 mice and cultured in DC medium. DC medium is Iscove's modified Dulbecco's medium (IMDM) supplemented with 10% FCS, 0.5 mM 2-ME, 4 mM L-glutamine, 50 μg/ml gentamicin, 5% of culture supernatant of murine GM-CSF transfected plamacytoma X63-Ag8 and 5% of culture supernatant of murine IL-4 transfected plamacytoma X63-Ag8 which contained approximately 10 ng/ml of GM-CSF and 10 ng/ml of IL-4 respectively. Culture medium was changed every three days.

Infection of dendritic cells and purification of MHC-bound peptides: A total of 4×10⁹ immature bone-marrow derived DCs were used for each experiment. Briefly, DCs were infected with C. muridarum at a 1:1 multiplicity of infection for 24 hr. As a control, DCs were incubated with LPS (1 microgram/ml; Sigma)

DCs treated with C. muridarum or LPS (as a control) were solubilized in lysis buffer [1% CHAPS, 150 mM NaCl, 20 mM Tris-HCl pH 8, 0.04% Sodium azide]. Protease inhibitors (Sigma) were added to minimize peptide degradation. MHC molecules (class I and class II) from Chlamydia-loaded and LPS-treated DCs were isolated using allele-specific anti-MHC monoclonal affinity columns (Table 1). The purified MHC molecules were washed and the peptides were eluted with 0.2N acetic acid and separated from high molecular weight material by ultrafiltration through 5-kDa cut-off membrane (Cox et al. 1997. The application of mass spectrometry to the analysis of peptides bound to MHC molecules in MHC— A practical Approach, pp. 142-160).

Identification of MHC-bound peptides: The purified MHC-bound peptides were analyzed using a linear trapping quadrupole/Fourier transform ion cyclotron resonance mass spectrometer (LTQ-FT, Thermo Electron) on-line coupled to Agilent 1100 Series nanoflow HPLCs using nanospray ionization sources (Proxeon Biosystems, Odense, Denmark). Analytical columns were packed into 15 cm long, 75 mm inner diameter fused silica emitters (8 mm diameter opening, pulled on a P-2000 laser puller from Sutter Instruments) using 3 mm diameter ReproSil Pur C₁₈ beads. LC buffer A consisted of 0.5% acetic acid and buffer B consisted of 0.5% acetic acid and 80% acetonitrile. Gradients were run from 6% B to 30% B over 60 minutes, then 30% B to 80% B in the next 10 minutes, held at 80% B for five minutes and then dropped to 6% B for another 15 minutes to recondition the column. The LTQ-FT was set to acquire a full range scan at 25,000 resolution in the FT, from which the three most intense multiply-charged ions per cycle were isolated for fragmentation in the LTQ. At the same time selected ion monitoring scans in the FT were carried out on each of the same three precursor ions. Fragment spectra were extracted using DTASuperCharge (available online from MSQuant Sourceforge—http://msquant.sourceforge.net and described in Mortensen et al 2009 J. Proteome Research 9:393-403) and searched using the Mascot algorithm against a database comprised of the protein sequences from mouse (self) and Chlamydia.

TABLE 1
Anti-MHC Monoclonal Antibodies Used for MHC Purification.

| MHC type | mAb Designation | ATCC# |
|---|---|---|
| Class I: H-2K^{b} | AF6-88.5.3 | HB-158 |
| Class I: H-2D^{b} | 20-80-4S* | HB-11 |
| Class II: I-A^{b} | Y-3P | HB-183 |

Interferon (IFN)-gamma assay of T-cell response to specific peptides: The Chlamydia peptides that associated with the class II MHC molecules were examined for recognition by Chlamydia specific CD4 T cells in vitro. Peptides corresponding to the sequences of each of the eight class II epitopes (SEQ ID NOs: 10-17) were synthesized and purified to 54-74% (Sigma Corporation) and then resolubilized in dimethyl sulfoxide at a concentration of 4 mg/mL. Immature DCs were generated and following maturation with LPS (1 microgram/ml), DCs were incubated for 4 hr with 10 microgram/int peptide. Chlamydia-specific CD4 T cells were generated by infecting C57BL/6 mice with C. muridarum as described in Rey-Ladino et al. 2005. Infec Immun 73:1568-1577. Briefly, spleens were isolated from naïve or mice that had cleared a C. muridarum infection, and CD4+ T cells were isolated with a MACS CD4+ T-cell isolation kit (Miltenyi Biotech). Peptide-pulsed DCs and CD4 T cells were co-cultured at a ratio of 1:3 and IFN-gamma production was determined from the culture supernatant following 48 hr incubation by ELISA (Pharmingen) as described (Rey-Ladino et al. 2005. Infec Immun 73:1568-1577). The amount of IFN-gamma present in the supernatants was used as a measure of antigen-specific T-cell recognition.

Delivery of Chlamydia MHC class II binding peptides by ex vivo pulsed DCs: The peptides (SEQ ID NOs: 10-17) derived from the Chlamydial proteins (PmpG, PmpF, L6 ribosomal protein, 3-oxoacyl-(acyl carrier protein) reductase, glyceraldehydes-3-phosphate dehydrogenase, ATP-dependent Clp protease, anti-anti-sigma factor the hypothetical protein TC0420) were pooled and used to pulse LPS-matured BMDCs for 4 h at 37° C. The peptide-pulsed DCs were washed three times and 1×10⁶ cells were adoptively transferred intravenously to naïve C57BL/6 mice and this process was repeated one week later. As a control, one group of mice received LPS-matured DCs that had not been treated with peptides (DC alone). One week following the final adoptive transfer, the mice were infected intranasally with 2000 IFU of C. muridarum and body weight was monitored every 48 hours post-infection.

Chlamydia strains: C. muridarum strain Nigg (mouse pneumonitis strain) was cultured in Hela 229 cells and elementary bodies (EBs) were purified by discontinuous density gradient centrifugation and stored at −80° C. as previously described in Hansen et al. 2008 J Infect Dis 198:758-767. The infectivity of purified EBs was titrated by counting Chlamydia inclusion forming units (IFUs) on HeLa cell monolayer with anti-EB mouse polyclonal antibody followed by biotinylated anti-mouse IgG (Jackson ImmunoResearch) and a DAB substrate (Vector Laboratories).

Cloning the Chlamydial protein antigens: The proteins containing the MHC II binding Chlamydia peptides (SEQ ID NOs: 10-17) were cloned, expressed and purified as follows: rplF, fabG, aasf, pmpG-1, TC0420, clp-1, pmpE/F-2 and gap DNA fragments were generated by PCR using genomic DNA isolated from C. muridarum. The PCR products were purified and cloned into either pGEX-6P-3 (GE Healthcare) for rplF, fabG, aasf, TC0420, and clp-1 or pET32a (Novagen) for pmpG-1, pmpE/F-2 and gap after restriction enzyme digestion with BamHI/NotI using standard molecular biology techniques. For pmpG-1, pmpE/F-2, only the first half of the gene (pmpG-1₂₅₋₅₀₀, pmpE/F-2₂₅₋₅₇₅ encoding amino acids 25-500 and 25-575 of PmpG-1 and PmpE/F-2, respectively) was cloned into the vector for expression. The sequences of the sub-cloned genes were confirmed by sequencing with dye-labeled terminators using the ABI PRISM kit (PE Biosystems). Plasmids containing the rplF, fabG, aasf, pmpG-1₂₅₋₅₀₀, TC0420, clp-1, pmpE/F-2₂₅₋₅₇₅ and gap sequences were transformed into the E. coli strain BL21(DE3) (Stratagene) where protein expression was carried out by inducing the lac promoter for expression of T7 RNA polymerase using isopropyl-beta-D-thiogalactopyranoside. The expressed RplF, FabG, Aasf, TC0420, and Clp-1 proteins with N-terminal GST-tag were purified from E. coli lysates by affinity chromatography using glutathione sepharose 4 fastflow purification system (GE Healthcare). PmpE-1₂₅₋₅₀₀, PmpE/F-2₂₅₋₅₇₅ and Gap proteins with N-terminal His-tag were purified by nickel column using the H is bind purification system (Qiagen). LPS removal was carried out by adding 0.1% Triton-114 in the wash buffers during purification.

Transfection of dendritic cells with Chlamydia proteins: After an 8-day culture, dendritic cells (DCs) were harvested for transfection with Chlamydia protein antigens. Approximately 65˜70% percent of the cell preparation were DCs as judged by a staining with anti-CD11c monoclonal antibody. DCs harvested on day 8 were washed twice in RPMI 1640. Sixty microlitres of the liposomal transfection reagent N-[1-(2,3-dioleoyloxy)propyl]-N,N,N-trimethylammonium methyl-sulfate (DOTAP; Roche) and individual or combination of Chlamydia proteins PmpG-1₂₅₋₅₀₀ (amino acids 25-500 of PmpG-1), RplF, PmpE/F-2₂₅₋₅₇₅ (amino acids 25-575 of PmpE/F-2), MOMP or the negative control protein, GST were mixed with 240 μl RPMI 1640 at room temperature in polystyrene tubes for 20 min. The final concentration of PmpG-1₂₅₋₅₀₀, PmpE/F-2₂₅₋₅₇₅, MOMP or GST protein in the DOTAP/protein mixtures is 0.2 mg/ml and RplF protein is 0.8 mg/ml. DCs (2˜3×10⁷) in 3 ml RPMI 1640 were added to the DOTAP/protein mixtures. The protein-transfected DCs were incubated for 3 h at 37° C., washed twice, resuspended in DC medium and then cultured overnight in the presence of 0.25 μg/ml LPS for maturation. DCs on day 8 pulsed with live EB (MOI:1) for 24 hours was prepared as a positive control. Antigen loaded DCs were used for in vitro immunohistochemical analysis and in vivo immunization.

Immunohistochemistry: The protein-transfected DCs were deposited onto Micro Slides using Shandon Cytospin (Thermo Electron Corp.). The DCs on the slides were fixed for 20 min in 4% paraformaldehyde in PBS. Subsequently, they were permeabilized for 10 min in 0.5% Triton X-100 in PBS. The cells were blocked for 20 min with PBS containing 1% horse serum, and incubated with corresponding antigen-specific polyclonal murine serum (1:200) respectively for 2 h. All anti-Chlamydia protein polyclonal antibodies (PmpG-1₂₅₋₅₀₀, RplF, PmpE/F-2₂₅₋₅₇₅, or MOMP) were made in our laboratory as follows: Balb/c mice were immunized three times subcutaneously with 10 μg recombinant Chlamydia protein formulated with Incomplete Freunds Adjuvant (Sigma). Two weeks after the final immunization, sera from each group were collected and pooled. All anti-Chlamydia protein polyclonal antibodies had titers ≧1:500,000 dilution as determined by ELISA. Biotinylated horse anti-mouse IgG (1:2000) (Vector Laboratories) was added and then the cells were incubated again for 1 h. Finally, the cells were incubated for 45 min with ABC Reagent (Vector Laboratories) and incubated with peroxidase substrate solution (DAB substrate kit SK-4100; Vector Laboratories) until the desired stain intensity developed. The slides were rinsed in tap water, counterstained with 0.1% toluidine blue, and again rinsed in tap water. All incubations were performed at room temperature and the slides were washed in PBS three times between incubations.

ELISA: CD4 T cells were isolated from the spleens of mice immunized i.p. with Chlamydia (14) or naive mice using MACS CD4 T cell isolation kit (Miltenyi Biotec). CD4 T cells of at least 90% purity were obtained as measured by FACS (data not shown). Purified BMDCs were cultured in a 96-well plate at 2×10⁵ cells/well and matured with LPS (1 microgram/ml) overnight, followed by treatment with 2 microgram/ml Chlamydia peptides or control peptides for 4 h, at which point the cells were washed to remove unbound peptides. After a 48-h coculture with CD4 T cells (5×10⁵/well), supernatants were collected and the production of IFN-gamma in the supernatants was determined by ELISA as described in Rey-Ladino et al., 2005 (supra).

ELISPOT assay: For the IFN-gamma ELISPOT assay, 96-well MultiScreen-HA filtration plates (Millipore) were coated overnight at 4 C with 2 μg/ml of murine IFN-gamma specific monoclonal antibody (BD PharMingen, Clone R⁴-6A2). Splenocytes isolated from mice in AIM-V medium were added to the coated plates at 10⁶ cells per well in presence of individual Chlamydia peptide at 2 μg/ml or individual Chlamydia protein at 1 μg/ml. After 20 h incubation at 37° C. and 5% CO₂, the plates were washed and then incubated with biotinylated murine IFN-gamma specific monoclonal antibodies (BD PharMingen, Clone XMG1.2) at 2 μg/ml. This was followed by incubation with streptavidin-alkaline phosphatase (BD PharMingen) at a 1:1000 dilution. The spots were visualized with a substrate consisting of 5-bromo-4-chloro-3-indolyl phosphate and nitro blue tetrazolium (Sigma-Aldrich).

Adoptive transfer of DCs transfected with Chlamydia protein antigens: Mice were vaccinated three times with a 2-week interval, intravenously (i.v.) into the tail vein with 1×10⁶ DCs transfected with Chlamydia protein PmpG-1₂₅₋₅₀₀, RplF, PmpE/F-2₂₅₋₅₇₅ or MOMP in 200 μl of PBS. DCs pulsed with live EB or with GST protein were used as positive and negative controls, respectively. Two weeks after the last immunization, six mice of each group were euthanized to isolate splenocytes for IFN-gamma ELISPOT assay. The remaining mice were used for Chlamydia infection challenge.

Pulmonary and cervicovaginal challenge and determination of Chlamydia titers: Two weeks after the final immunization, five to ten mice from each group were intranasally challenged with 2000 IFU of C. muridarum. Weight loss was monitored each or every two days. On 10 day after intranasal challenge, the mice were euthanized and the lungs were collected for Chlamydia titration. Single-cell suspensions were prepared by homogenizing the lungs with tissue grinders and coarse tissue debris was removed by centrifugation at 1000×g for 10 min at 4° C. The clarified suspensions were serially diluted and immediately inoculated onto HeLa 229 monolayers for titration. For genital tract infections, one week after the final immunization, ten mice from each group were injected subcutaneously with 2.5 mg of medroxyprogesterone acetate (Depo-Provera; Pharmacia and Upjohn). One week after Depo-Provera treatment, the mice were challenged intravaginally with 1500 IFU of C. muridarum. Cervicovaginal washes were taken at day 6 and day 13 after infection and stored at −80° C. for titration on HeLa cells as described previously in Bilenki et al. 2005 J Immunol 175:3197-3206.

Adjuvants: Three adjuvants (CpG ODN 1826, AbISCO-100, and Dimethyldioctadecylammonium Bromide/D-(+)-trehalose 6,6′-dibenhate (DDA/TDB) were studied in the present study. CpG ODN 1826 (5′-TCCATGACGTTCCTGACGTT-3′, phosphorothioate modified, Integrated DNA Technologies, Inc.) (SEQ ID NO: 48) was used as either a free form (Free CpG) or a form conjugated with liposomal nanoparticle (LN-CpG). AbISCO-100 adjuvant (ISCONOVA Sweden) is a selection of purified fractions of quillaja saponins formulated with a mixture of cholesterol (ovine wool) and phosphatidyl choline (egg). DDA Dimethyldioctadecylammonium Bromide (product No. 890810P) and TDB D-(+)-trehalose 6,6′-dibehenate (product No. 890808P) were purchased from Avanti Polar Lipids (Alabaster Ala.). For DDA/TDB formulation, DDA was mixed into 10 mM Tris-buffer at pH 7.4 to a concentration of 1.67 mg/ml, heated to 80° C. while being stirred continuously on a magnetic hot plate for 20 min, and then cooled to room temperature. TDB was suspended in dH₂O containing 2% dimethyl sulfoxide to a concentration of 5 mg/ml by repeated passaging through a fine-tipped pipette followed by 30 seconds of vortexing. This step was repeated three times before freezing the solution at −20° C. until use. 5 ml TDB (1 mg/ml) was added into 15 ml DDA (1.67 mg/ml). The resulting solution was then vortexed briefly and stored at 4° C. until use. The final concentration of DDA was 1.25 mg/ml and TDB was 0.25 mg/ml. Each inoculation dose, 200 μl for immunization contained 250 μg DDA and 50 μg TDB.

Mouse immunization: Four mouse trials were conducted in this study. All mice except the live EB group were immunized three times subcutaneously (sc) in the base of tail at 2 week intervals. Mice intranasally infected with 1500 inclusion-forming units (IFU) live C. muridarum (EB) were set up as positive controls.

In the first trial, groups of six C57/BL6 mice were immunized with 20 μg Chlamydia protein (PmpG-1 or MOMP) mixed with 700 μg LN-CpG or 700 μg free CpG. Groups of LN-CpG alone and PBS immunization were set up as negative controls. In the second trial, groups of eight C57/BL6 mice were immunized with 5 μg individual Chlamydia proteins PmpG-1, PmpE/F-2, MOMP or a combination (1.67 μg for each protein) formulated with AbISCO-100 (12 μg) or DDA/TDB (250 μg DDA, 50 μg TDB) as follows: (1) PmpG-1+AbISCO-100 (PmpG+AbISCO); (2) PmpE/F-2+AbISCO-100 (PmpF+AbISCO); (3) MOMP+AbISCO-100 (MOMP+AbISCO); (4) PmpG-1+PmpE/F-2+MOMP+AbISCO-100 (G+F+M+AbISCO); (5) AbISCO-100 alone (AbISCO alone); (6) PmpG-1+DDA/TDB (PmpG+DDA/TDB); (7) PmpE/F-2+DDA/TDB (PmpF+DDA/TDB); (8) MOMP+DDA/TDB; (9) PmpG-1+PmpE/F-2+MOMP+DDA/TDB (G+F+M+DDA/TDB); (10) DDA/TDB alone; (11) PBS; or (12) EB. In the third trial, three groups of eight BALB/c mice were immunized as follows: (1) G+F+M+DDA/TDB, (2) DDA/TDB alone; (3) EB. The mice in above three animal trials were then challenged with live EB for protection and pathology evaluation.

In the fourth trial, groups of sixteen C57/BL6 mice were immunized with 5 μg PmpG-1 formulated with DDA/TDB (250 μg DDA, 50 μg TDB), AbISCO-100 (12 μg) or CpG (20 μg). Two weeks after the last immunization, half of the mice in each group were sacrificed to isolate splenocytes for lymphocyte multi-color flow cytometry, ELISA and enzyme-linked immunospot (ELISPOT) assays; the other half of the mice were challenged with live EB and sacrificed seven days later to isolate splenocytes and iliac lymph nodes for multi-color flow cytometry.

Genital tract infection and determination of Chlamydia titer: One week after the last immunization, mice were injected s.c. with 2.5 mg of medroxyprogesterone acetate (Depo-Provera; Pharmacia and Upjohn). One week after Depo-Provera treatment, mice were challenged intravaginally with 1500 IFU of C. muridarum. Cervicovaginal washes were taken atselected dates after infection and stored at −80° C. for titration on HeLa cells as described (Bilenki et al., 2005. J. Immunol. 175:3197-3206).

Cytokine measurement: The culture supernatants of the splenocytes stimulated with PmpG-1 protein or HK-EB for 48 hours were collected and analyzed with respect to TNF-α production with a sandwich ELISA using corresponding specific capture and detection antibodies (BD PharMingen). TNF-α levels were calculated using standard curve constructed by recombinant murine TNF-α (BD PharMingen).

Multiparameter flow cytometry: Two weeks after the last immunization or seven days after live EB challenge, the mice from specified groups were sacrificed and the cells harvested from spleen and iliac lymph nodes (after challenge) were stimulated with 2 μg/ml antibody to CD28 and PmpG-1 protein (1 μg/ml) or HK-EB (5×105 IFU/ml) in complete RPMI 1640 for 4 h at 37° C. Brefeldin A was added at a final concentration of 1 μg/ml and cells were incubated for an additional 12 h before intracellular cytokine staining. Cells were surface stained for CD3, CD4 and CD8 as well as the viability dye, red-fluorescent reactive dye (RViD) (L23102, Molecular Probes) followed by staining for IFN-γ, TNF-α and IL-17 using the BD Cytoperm kit according to the manufacturer's instruction. Finally, the cells were resuspended in 4% formaldehyde solution. All antibodies and all reagents for intracellular cytokine staining were purchased from BD Pharmingen except where noted. We acquired 200,000 live lymphocytes per sample using an Aria flow cytometer and analyzed the data using FlowJo software (Tree Star).

Evaluation of mouse genital tract tissue pathologies: Mice were sacrificed 60 days after challenge and the mouse genital tract tissues were isolated. Hydrosalpinx in only one (unilateral) or both (bilateral) oviducts were visually identified as the pathologic outcome in the vaccine groups.

Statistical analysis: All data were analyzed with the aid of a software program (GraphPad Prism 3.0). Differences between the means of experimental groups were analyzed using an independent, two-tailed t-test at the level of p<0.05.

### Example 1

### Identification of MHC Class II (I-Ab)-Bound Chlamydial Peptides

The purified MHC-bound peptides were identified by tandem mass spectrometry. In total 318 MHC Class II (1-Ab)-bound peptides were isolated. Many of these peptides were derived from the same epitope, with varying degrees of proteolytic processing and 157 distinct epitopes were isolated from 137 unique source proteins. As determined by BLAST identification of the peptides using the National Centre for Biotechnology Information database (GenPept), four peptides were derived from the Chlamydia L6 ribosomal protein (RplF), two peptides from the 3-oxoacyl-(acyl carrier protein) reductase, two peptides from polymorphic membrane protein G (PmpG), one peptide from polymorphic membrane protein E/F (PmpE/F), one peptide from glyceraldehydes-3-phosphate dehydrogenase, one peptide from ATP-dependent Clp protease, one peptide from the anti-anti-sigma factor and one peptide from a hypothetical protein TC0420, all from the C. muridarum proteome (Table 2).

### Example 2

### Identification of MHC Class I (H2-Kb)-Bound Peptides

One of the H2-Kb-bound peptides that was isolated (SEQ ID NO: 19), corresponded to an amino acid permease from the C. muridarum proteome. The 79 remaining peptides that were isolated with were self-peptides.

TABLE 2
MHC-bound peptides

| | | | C.trachomatis |
|---|---|---|---|
| | Protein | Peptide sequence | Accession |
| 19 | Amino acid | SSLFLVKL | NP_219919 |
| | permease | | |
| 20 | Ribosomal | GNEVFVSPAAHIID | AAC68115 |
| | protein L6 | | |
| 21 | Ribosomal | GNEVFVSPAAHIIDRPG | AAC68115 |
| | protein L6 | | |
| 22 | Ribosomal | KGNEVFVSPAAHIIDRPG | AAC68115 |
| | protein L6 | | |
| 23 | Ribosomal | EVFVSPAAHIIDRPG | AAC68115 |
| | protein L6 | | |
| 24 | 3-oxoacyl-(acyl | SPGQTNYAAAKAGIIG | NP_219742 |
| | carrier protein) | | |
| | reductase | | |
| 25 | 3-oxoacyl-(acyl | SPGQTNYAAAKAGIIGFS | NP_219742 |
| | carrier protein) | | |
| | reductase | | |
| 26 | Anti-anti-sigma | KLDGVSSPAVQESISE | NP_219936 |
| | factor | | |
| 27 | Polymorphic | SPIYVDPAAAGGQPPA | AAC68469 |
| | membrane | | |
| | protein G1 | | |
| | family | | |
| 28 | Polymorphic | ASPIYVDPAAAGGQPPA | AAC68469 |
| | membrane | | |
| | protein G1 | | |
| | family | | |
| 29 | Hypothetical | DLNVTGPKIQTDVD | NP_219646 |
| | protein (CT143) | | |
| 30 | ATP-dependent | IGQEITEPLANTVIA | NP_220225 |
| | Clp protease | | |
| 31 | Polymorphic | FHLFASPAANYIHTGP | AAC68468 |
| | membrane | | |
| | protein E/F | | |
| | family F-2 | | |
| 32 | Glyceraldehyde | MTTVHAATATQSVVD | NP_220020 |
| | 3-phosphate | | |
| | dehydrogenase | | |

### Example 3

### Recognition of Chlamydial Peptides and Production of Interferon Gamma by Immune CD4+ T-Cells

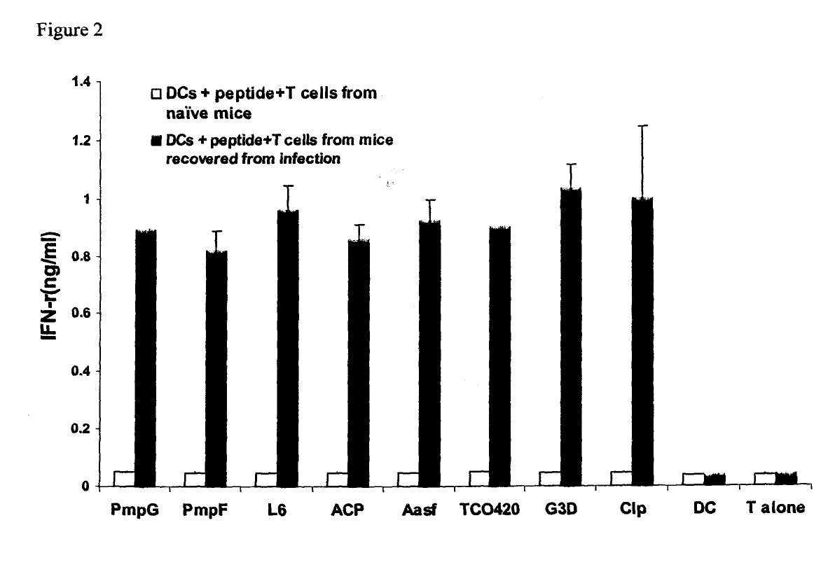

Peptides comprising the identified MHC class II Chlamydia epitopes were synthesized (SEQ ID NOs: 10-17) and examined for recognition by Chlamydia specific CD4⁺ T cells in vitro. Briefly, CD4⁺ T cells from immune or naïve mice were co-cultured with peptide-pulsed DCs as described (Cohen et al., 2006. Journal of Immunology 176: 5401-5408). IFN-gamma production was determined from the culture supernatant following 48 hr incubation by ELISA. All the MHC class II Chlamydia peptides were recognized by Chlamydia-specific CD4 T cells as measured by antigen specific IFN-gamma production (FIG. 2), suggesting that these antigens are immunologically relevant and may be useful as antigens in Chlamydia vaccine development.

### Example 4

### Delivery of Chlamydia MHC class II Binding Peptides by Ex Vivo Pulsed DCs

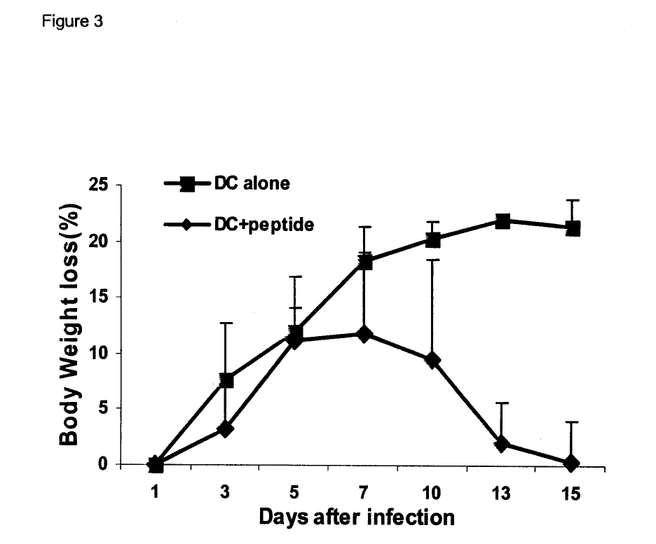

To evaluate whether the identified Chlamydia MHC class II peptides (SEQ ID NOs: 10-17) were able to protect mice against Chlamydia infection using a lung infection model, the peptides (SEQ ID NOs: 10-17) were synthesized, pooled together and used to load LPS-matured DCs ex vivo. The peptide-pulsed DCs were adoptively transferred intravenously to naïve C57BL/6 mice. As a control, another group of mice received LPS-matured DCs that had not been treated with peptides (DC alone). One week following the second adoptive transfer both groups of mice were infected intranasally with 2000 inclusion forming units (IFUs) of C. muridarum. Body weight was monitored every 48 hours post infection. Mice adoptively transferred with peptide-pulsed DCs (FIG. 3) reversed body weight loss by day 10 post-infection returning to their pre-infection body weight by day 15. In contrast, mice that had been adoptively transferred with LPS-matured non pulsed DCs failed to regain their starting body weight over this time.

### Example 5

### Identification of the Immunodominant Chlamydia Antigens Among the 8 MHC class II Binding Peptides

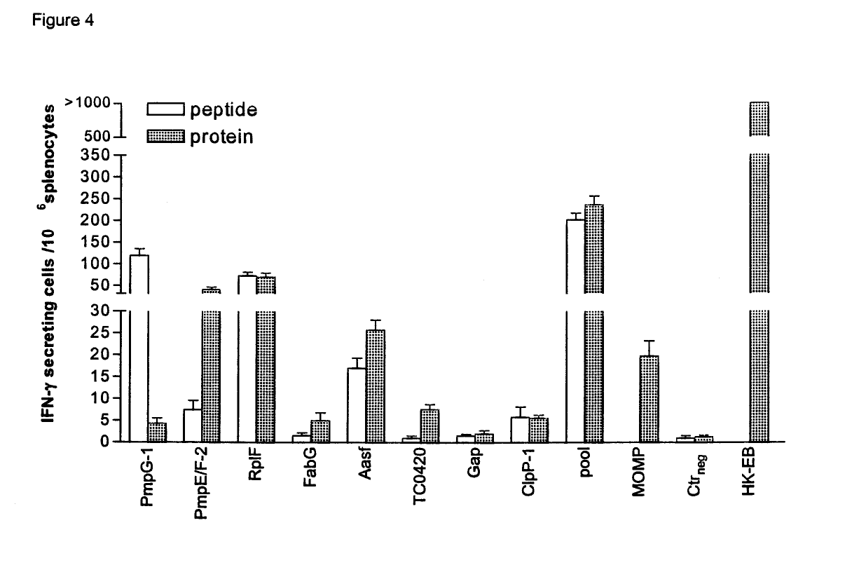

To determine which individual peptides or proteins are immunodominant in the context of natural infection, we performed IFN-gamma ELISPOT assays using splenocytes from C57BL/6 mice that had recovered from live C. muridarum infection. Splenocytes from mice harvested one month after C. muridarum infection were stimulated in vitro for 20 h with either 2 μg/ml of the individual peptide or pooled peptide epitopes (SEQ ID NOs: 10-17) or 1 μg/ml of the individual protein or with pooled proteins (RplF, FabG, Aasf, PmpG-1, TC0420, C1p, PmpE/F and Gap). Irrelevant OVA peptide and GST were used as peptide and protein negative controls respectively and heat killed EB (HK-EB) as positive control. Since MOMP has been long standing candidate in Chlamydia vaccine studies, MOMP was also set up as a reference antigen. As shown in FIG. 4, immune splenocytes exposed to HK-EB developed the highest numbers of IFN-gamma secreting cells where more than 1000 IFN-gamma-secreting cells were detected among 10⁶ splenocytes. In contrast, splenocytes stimulated with the OVA peptide or GST protein as negative controls showed nearly blank background levels indicating that IFN-gamma secreting cells detected in the experimental system are Chlamydia antigen-specific. Stimulation by pooled peptides or pooled proteins induced significantly higher numbers of IFN-gamma secreting cells than stimulation with individual Chlamydia antigens (p<0.05).

Immune splenocytes stimulated with individual Chlamydia antigens exhibited markedly different levels of IFN-gamma response (FIG. 4). The results demonstrated that IFN-gamma responses in immune splenocytes in response to stimulation with PmpG-1 peptide, PmpE/F-2₂₅₋₅₇₅ protein, RplF peptide (SEQ ID NO: 10) and RplF protein were strong. The response to the Aasf peptide (SEQ ID NO: 12), Aasf protein or MOMP protein were moderate and others were weaker. Thus, three of the eight antigens (PmpG-1, RplF and PmpE/F-2—SEQ ID NO: 10, 13 and 16) were determined as immunodominant based on their strong IFN-gamma responses by ELISPOT assay to stimulation by either the peptide or parent protein.

### Example 6

### Efficient Intracellular Uptake of Chlamydia Protein Antigens by DCs Using DOTAP as a Delivery System

Since protein antigens require endocytotosis and lysosomal processing before the peptide is loaded onto MHC class II molecules, the cationic liposome DOTAP was used to deliver the Chlamydia proteins intracellularly into DCs. The intracellular uptake of PmpG-1₂₅₋₅₀₀, PmpE/F-2₂₅₋₅₇₅, RplF or MOMP protein was visualized by immunohistochemistry following transfection (data not shown). Efficient uptake of PmpG-1₂₅₋₅₀₀, RplF, PmpE/F-2₂₅₋₅₇₅ and MOMP was detected in the cytoplasm of the Chlamydia protein-transfected DC, whereas no signal was detected in non-transfected DCs. Thus the cationic liposome DOTAP efficiently delivered Chlamydia protein intracellularly into DCs.

After DC transfection with Chlamydia proteins, DCs were matured with LPS for 18 h. The cell surface antigen expression on the transfected DCs was assessed after LPS stimulation. There was no phenotypic difference between DCs transfected with different Chlamydia antigens or GST (data not shown). DCs stimulated with LPS expressed enhanced levels of CD40, MHC class II and CD86 compared with unstimulated DCs. (data not shown).

### Example 7

### Specific Immune Responses to Chlamydia Antigens Following Adoptive Transfer of DCs Transfected with Chlamydia Proteins

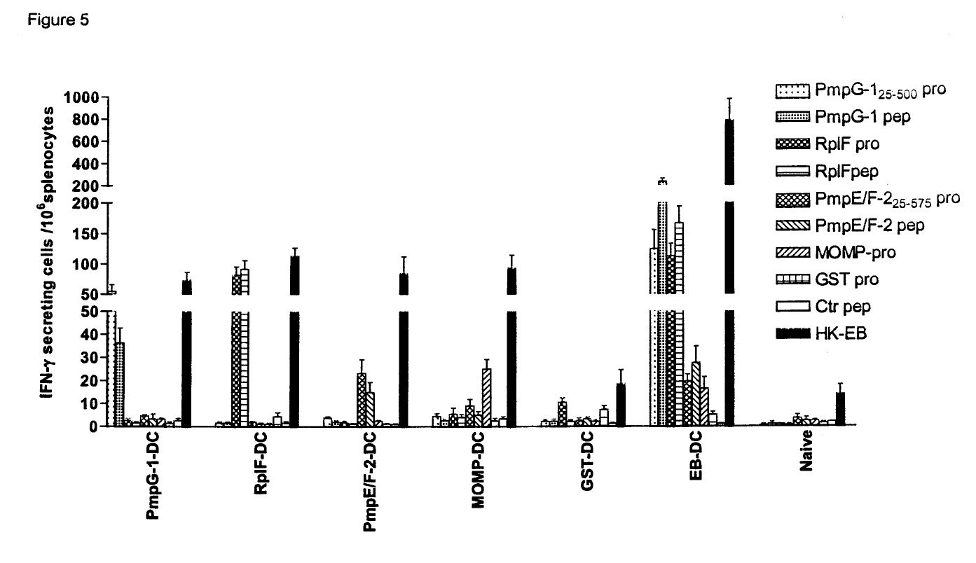

Mice were adoptively transferred with DCs that had been previously transfected with the immunodominant protein antigens. A group of DCs transfected with MOMP was set up as a reference control antigen. As a negative control, one group of mice received DCs pulsed with GST protein. As a positive control, another group of mice received DCs pulsed with viable C. muridarum EB. Two weeks following the final adoptive transfer, Chlamydia-specific immune responses in vaccinated mice were assessed by enumerating antigen-specific IFN-gamma producing cells in splenocytes from each group after exposure to Chlamydia antigens (FIG. 5). The results showed that the mice which received the DCs transfected with individual Chlamydia muridarum proteins (PmpG-1, RplF and PmpE/F-2) developed significant antigen specific IFN-gamma responses to the corresponding peptides and proteins but not to other non-related Chlamydia antigens. Importantly, mice immunized with DCs transfected with individual Chlamydia proteins demonstrated strong specific immune responses to HK EB (p<0.01). As a positive control, mice that received DCs pulsed with live C. muridarum (EB) developed the strongest IFN-gamma responses to HK-EB as shown by more than 1000 IFN-gamma-secreting cells detected among 10⁶ splenocytes. This group also exhibited strong antigen-specific IFN-gamma responses to PmpG-1 peptide (SEQ ID NO: 13) or PmpG-1 protein and RplF peptide (SEQ ID NO: 10) or RplF protein and moderate responses to PmpE/F-2 peptide (SEQ ID NO: 16) or PmpE/F-2 protein and MOMP. In contrast, naïve and GST-DC vaccinated splenocytes stimulated with the Chlamydia antigens or HK-EB showed low background levels except for the GST-DC group which exhibited some responses to GST protein and the GST-fusion protein, RplF. IL-4 ELISPOT assays were also performed and showed no or very low Chlamydia antigen specific IL-4 secretion in any groups immunized with DCs transfected with individual Chlamydia protein (data not shown).

### Example 8

### Adoptive Transfer of DCs Transfected with the PmpG-1, PmpE/F-2 or RplF Protein Antigens Leads to Protection Against Chlamydia Infection in Mice

To evaluate whether the Chlamydia protein antigens were able to protect mice against subsequent Chlamydia pulmonary or genital tract infection, we undertook adoptive transfer studies using LPS-matured DCs transfected ex vivo with either PmpG-1₂₅₋₅₀₀, RplF, PmpE/F-2₂₅₋₅₇₅ or MOMP. Mice received DCs transfected with GST or pulsed with viable C. muridarum were set up as negative and positive controls, respectively. Two weeks following the final adoptive transfer, mice were challenged intranasally or vaginally with C. muridarum.

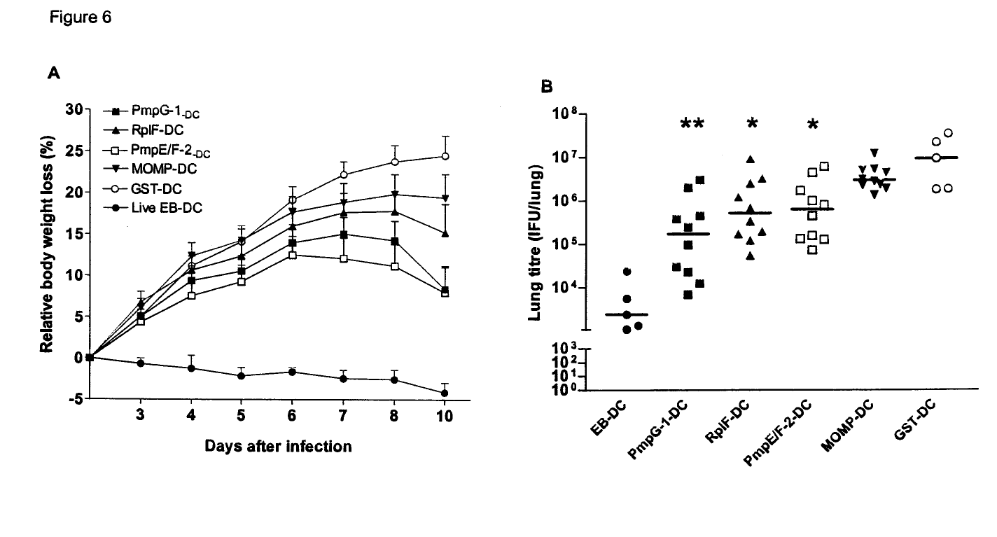

After the intranasal challenge, protection was measured by body weight loss and bacterial load in the lungs. As shown in FIG. 6A, mice adoptively immunized with live EB-pulsed DC demonstrated excellent protection against infection as indicated by no body weight loss. In contrast, mice immunized with GST-pulsed DC exhibited the largest weight loss. The mean body weight loss on day 10 post infection reached 24.4±2.4% in the negative control group (p<0.001 vs. positive control). Mice vaccinated with the individual DC that were transfected with individual Chlamydia muridarum protein antigens showed varying levels of protection as indicated by different degrees of body weight loss during the 10-day period. The mean relative body weight loss at day 10 in groups of PmpE/F-2-DC, PmpG-1-DC, RplF-DC, or MOMP was 7.9±3.1%, 8.1±2.7%, 15.2±3.4%, and 19.4±2.8% respectively.

Ten days after the intranasal challenge, lungs were harvested and Chlamydia inclusion forming units were determined by plating serial dilutions of homogenized lungs onto HeLa 229 cells (FIG. 6B). When compared to the negative control group, the median Chlamydia titers decreased 1.8 orders of magnitude (log₁₀) in mice vaccinated with PmpG-1-DC (p<0.01) and decreased 1.2 and 1.1 orders of magnitude in mice vaccinated with RplF-DC (p<0.05) and PmpE/F-2-DC (p<0.05) respectively. There was no statistically significant difference in lung Chlamydia titers between mice vaccinated with MOMP-DC and the negative control group.

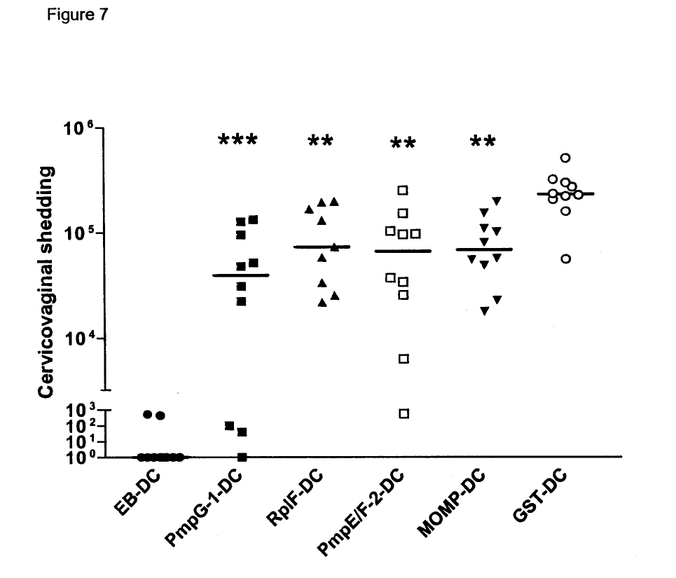

Protection against intravaginal infection was assessed by isolation of Chlamydia from cervicovaginal wash and determination of the number of IFU recovered from each experimental group at day 6 post-infection (FIG. 7). The results showed that the cervicovaginal shedding of C. muridarum in mice immunized with any of the four Chlamydia protein-transfected DCs was significantly lower than that of mice who received GST-transfected DCs (p<0.001 in PmpG-1 group; p<0.01 in RplF group; p<0.01 in PmpE/F-2 group; p<0.01 in MOMP group). Taken together, mice vaccinated with DCs transfected with Chlamydia protein PmpG-1₂₅₋₅₀₀, RplF or PmpE/F-2₂₅₋₅₇₅ polypeptides exhibited significant resistance to challenge infection as indicated by log₁₀ reduction in the median Chlamydia titer in comparison with the negative control group in both lung model and genital tract model. MOMP, as a reference antigen, conferred significant protection but only in the genital tract model. These data demonstrated that vaccination with DCs transfected with PmpG-1₂₅₋₅₀₀ polypeptide developed the greatest degree of protective immunity among the four Chlamydia antigens evaluated.

### Example 9

### Vaccination with both PmpG-1 and PmpE/F-2 Protein Antigens Leads to Synergistic Protection Against Chlamydia Infection in Mice

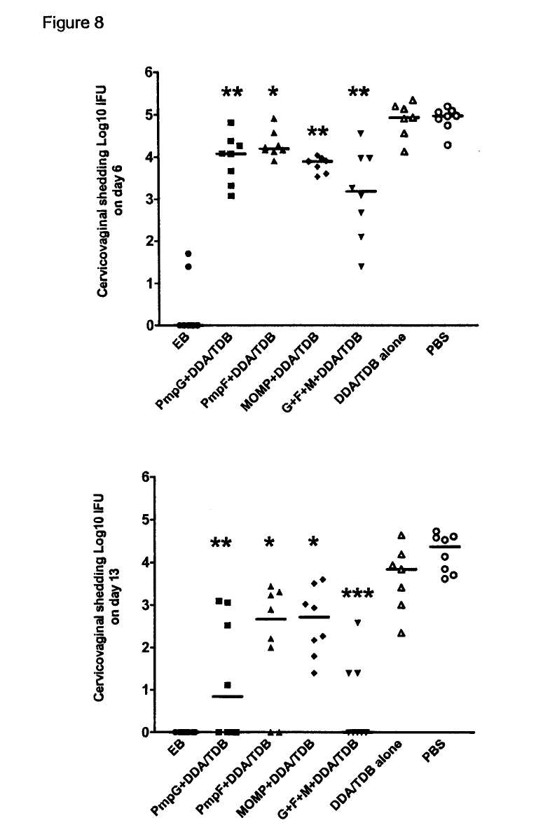

To evaluate whether combinations of Chlamydia protein antigens were able to protect mice against genital tract infection, we vaccinated mice with either PmpG-1₂₅₋₅₀₀, PmpE/F-2₂₅₋₅₇₅ or MOMP, or a pool of PmpG-1₂₅₋₅₀₀, PmpE/F-2₂₅₋₅₇₅ and MOMP, formulated with adjuvant DDA/TDB. C57BL/6 mice were vaccinated three times with a 2-week interval with PBS, DDA/TDB alone as negative controls and live Chlamydia EB as positive control. One week after the final immunization, the mice from each group were injected with Depo-Provera. One week after Depo-Provera treatment, the mice were infected intravaginally with 1500 IFU live C. muridarum. Protection against intravaginal infection was assessed by isolation of Chlamydia from cervicovaginal wash and determination of the number of IFU recovered from each experimental group at day 6 and day 13 post-infection (FIG. 8).

As shown in FIG. 8, mice immunized with EB demonstrated excellent protection against infection as indicated by large reductions in cervicovaginal shedding at 6 and 13 days post infection. In contrast, the negative control (DDA/TDB adjuvant alone) group of mice, showed very high levels of cervicovaginal shedding. When compared to the negative control group, the median cervicovaginal shedding decreased 1.0 and 2.9 orders of magnitude (log₁₀) in mice vaccinated with PmpG-1 (p<0.01, p<0.001) on day 6 and day 13. The bacterial titer decreased 0.8 and 1.1 orders of magnitude in mice vaccinated with PmpE/F-2 (p<0.05, p<0.05). Cervicovaginal shedding decreased 1.8 and 3.8 orders of magnitude in mice vaccinated with a cocktail containing PmpG-1 PmpE/F-2 and MOMP (p<0.01, p<0.001). MOMP, as a reference antigen, conferred significant protection at both 6 and 13 days post infection. Taken together, mice vaccinated with Chlamydia protein PmpG-1₂₅₋₅₀₀ and PmpE/F-2₂₅₋₅₇₅ exhibited significant resistance to challenge infection as indicated by reduction in the median Chlamydia titer in comparison with the adjuvant alone group in the genital tract model.

### Example 10

### Multiple Chlamydia Antigens Formulated with DDA/TDB Exhibit Protection Against Challenge with Live C. muridarum

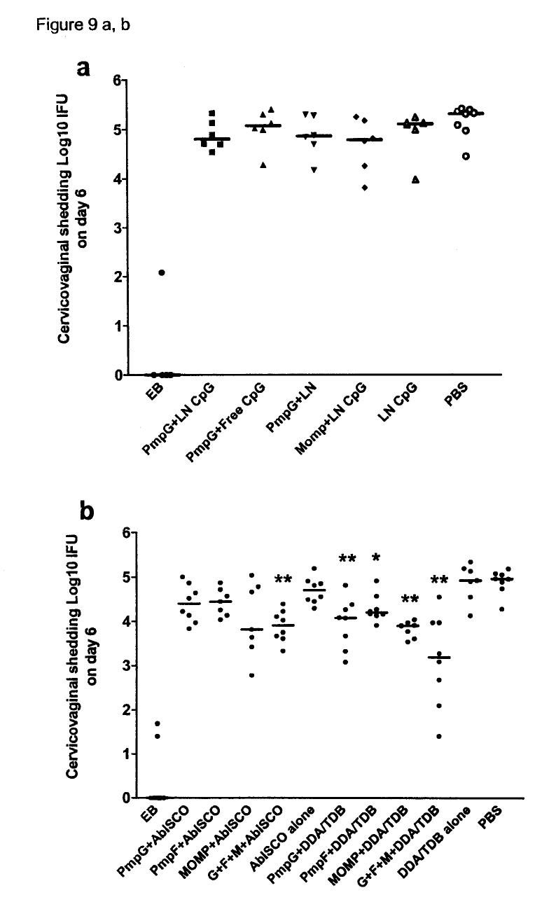

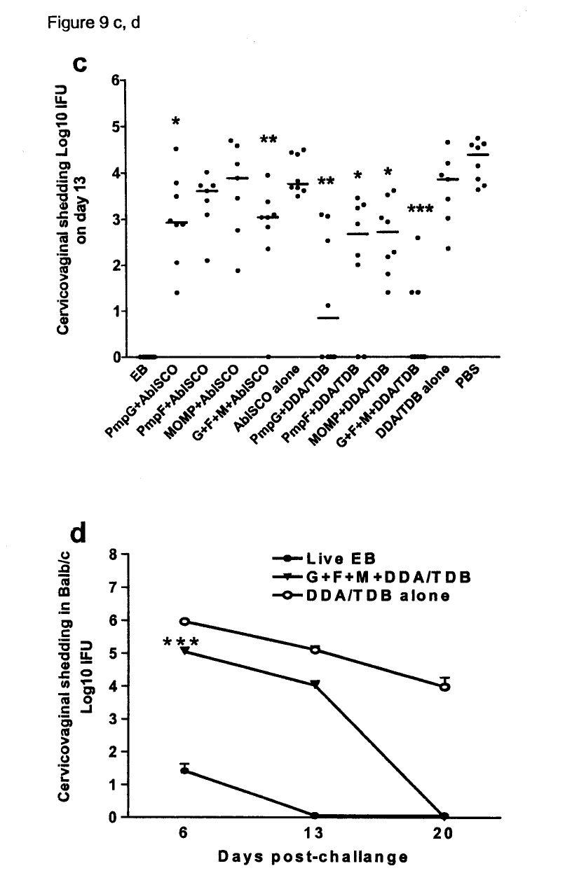

In order to discover a Th1-polarizing adjuvant that efficiently delivers Chlamydia antigens, we first tested mouse specific CpG-ODN 1826. In the current trial, mice were immunized with PmpG-1 or MOMP protein formulated with either a free form of CpG ODN 1826 (Free CpG) or a liposomal nanoparticle conjugated form (LN-CpG). Mice immunized with PmpG-1 plus liposomal nanoparticle only (PmpG+LN), LN-CpG only or PBS were set up as negative controls and mice recovered from previous intranasal infection served as a positive control. Two weeks after the final immunization, mice were challenged vaginally with C. muridarum. Protection against intravaginal infection was assessed by isolation of Chlamydia from cervicovaginal wash and the determination of the number of IFU recovered from each experimental group at day 6 post-infection. As shown in FIG. 9a, mice immunized with live EB exhibited excellent protection against infection, as indicated by no or very low Chlamydia detection. However, the cervicovaginal shedding of C. muridarum in all other groups did not have any significant difference (FIG. 9a), demonstrating that CpG ODN formulated with PmpG-1 or MOMP failed to induce protection against Chlamydia infection.

In the next trial we evaluated protection against Chlamydia infection in C57 mice immunized with individual PmpG-1, PmpE/F-2, MOMP protein or a combination formulated with adjuvant AbISCO-100 or DDA/TDB. After the genital challenge, we tested the Chlamydia inclusion titers in cervicovaginal washes taken at day 6 and day 13. The results indicate that DDA/TDB exhibited overall better protection than AbISCO. As shown in FIG. 9b, mice immunized with individual PmpG-1, PmpE/F-2, MOMP protein or a combination formulated with DDA/TDB demonstrated significant reduction of Chlamydia titer at day 6 when compared to DDA/TDB adjuvant alone group (p<0.01 in the PmpG+DDA/TDB group, p<0.05 in the PmpF+DDA/TDB group, p<0.01 in the MOMP+DDA/TDB group and p<0.01 in the G+F+M+DDA/TDB group). The antigen combination group tended to develop higher protection than individual antigen group, as indicated by much lower Chlamydia titers detected in some mice of the G+F+M+DDA/TDB group. Significant protection induced by AbISCO was only observed in the combination group (p<0.01 vs AbISCO alone), but not in the individual antigen group. Data at day 13 (FIG. 9c) further confirmed the results obtained at day 6. Mice immunized with individual PmpG-1 protein or the combination of three Chlamydia proteins formulated with AbISCO exhibited significant protection at day 13 compared to the adjuvant alone group (p<0.05 in the PmpG+AbISCO group and p<0.01 in the G+F+M+ AbISCO group). On the other hand, all DDA/TDB formulated-Chlamydia antigens conferred significant protection at day 13 when compared to DDA/TDA alone group and vaccination with G+F+M+DDA/TDB exhibited the greatest degree of protective immunity among all the groups tested. Of interest, five out of eight mice vaccinated with G+F+M+DDA/TDB completely resolved the infection and the other three mice in this group showed very low Chlamydia load at day 13 (p<0.01 in the PmpG+DDA/TDB group, p<0.05 in the PmpF+DDA/TDB group, p<0.05 in the MOMP+DDA/TDB group and p<0.001 in the G+F+M+DDA/TDB group).

Since all the protection results obtained above were observed in C57BL/6 mouse, the strain in which the antigens were originally discovered by immunoproteomics, we challenged mice with a different MHC genetic background to determine if immunization with multiple Chlamydia protein antigens and DDA/TDB conferred protection. BALB/c mice were immunized with G+F+M+DDA/TDB or DDA/TDB alone, and mice infected with live EB were set up as a positive control. Chlamydia inclusion titers in the cervicovaginal washes were detected post-challenge. As shown in FIG. 9d, BALB/c mice immunized with live EB demonstrated excellent protection against infection, as indicated by very low bacterial load at day 6, and no Chlamydia detected at day 13 and day 20. Vaccination with G+F+M+DDA/TDB in BALB/c mice significantly decreased the Chlamydia load in the cervicovaginal washes at all three selected dates when compared with DDA/TDB alone (p<0.001). At day 20 after challenge, all BALB/c mice vaccinated with G+F+M+DDA/TDB completely resolved infection.

Collectively, among the three tested adjuvants CpG ODN 1826, AbISCO-100 and DDA/TDB, CpG ODN formulation was not able to engender protection against Chlamydia infection at any level in vaccinated mice. The AbISCO formulation conferred moderate protection while the DDA/TDB formulation showed the greatest efficacy. The combination of PmpG-1, PmpE/F-2 and MOMP formulated with DDA/TDB generated a synergistic effect that exhibited the greatest degree of protective immunity among all groups studied. Moreover, G+F+M+DDA/TDB vaccination also stimulated significant protection in BALB/c mice with a different MHC background from C57BL/6 mice.

### Example 11

### PmpG-1 Formulated with DDA/TDB Induced Strong IFN-γ, TNF-α and IL-17 Responses Characterized by a High Frequency of IFN-γ/TNF-α and IFN-γ/IL-17 Double Positive CD4+ T Cells in Immunized Mice

In order to explore the cellular mechanisms for different degrees of protection induced by the three adjuvants, C57BL/6 mice were immunized with PmpG-1 formulated with DDA/TDB, AbISCO-100 and CpG ODN 1826 and then challenged with live C. muridarum. The magnitude and quality of T cells producing IFN-γ, TNF-α and IL-17 were assessed before and after challenge using ELISPOTs, ELISA and multiparameter flow cytometry.

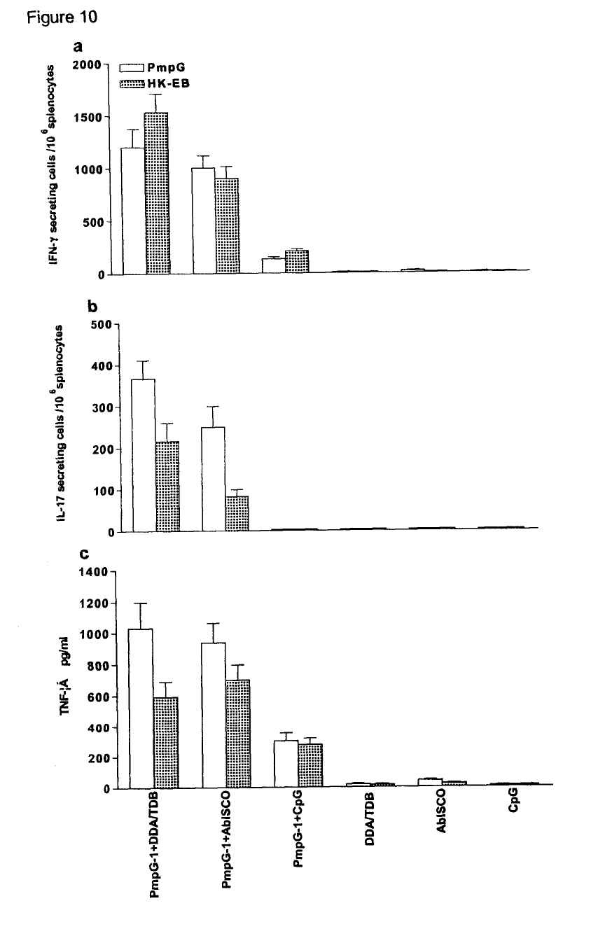

In this study, the ELISPOTs assay was performed to detect IFN-γ and IL-17 producing cells in immune splenocytes stimulated with PmpG-1 protein or HK-EB. ELISA was performed to measure TNF-α level in the supernatant of stimulated immune splenocytes. Splenocytes after immunization with PmpG-1 formulated with DDA/TDB, AbISCO-100 or CpG ODN 1826 exhibited markedly different levels of IFN-γ (FIG. 10a), TNF-α (FIG. 10c) and IL-17 response (FIG. 10b). The PmpG+DDA/TDB immune splenocytes exposed to either PmpG-1 protein or HK-EB developed the highest numbers of IFN-γ, and IL-17-secreting cells; the PmpG+AbISCO immune splenocytes demonstrated less strong IFN-γ and IL-17 responses but similar levels of TNF-α when compared with PmpG+DDA/TDB immunization; and the weakest IFN-γ, TNF-α response and no IL-17 response were induced by the PmpG+CpG immunization. In addition, splenocytes from adjuvant alone immunized mice which served as negative controls showed nearly blank background levels, indicating that cytokine responses detected in the experimental system are Chlamydia Ag-specific. The varying levels of IFN-γ and IL-17 response in mice immunized with different adjuvants are remarkably consistent with the degree of protection against challenge infection (FIG. 9) suggesting that a correlate of vaccine-mediated protection against Chlamydia is the magnitude of specific cytokine responses.

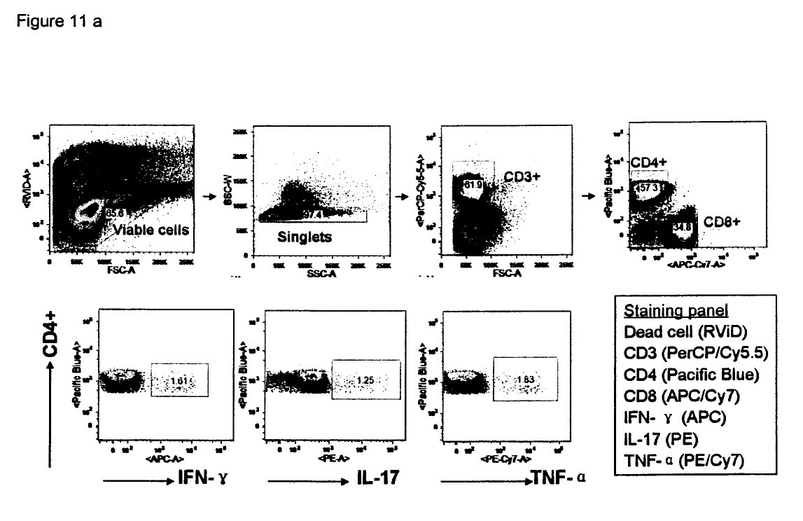

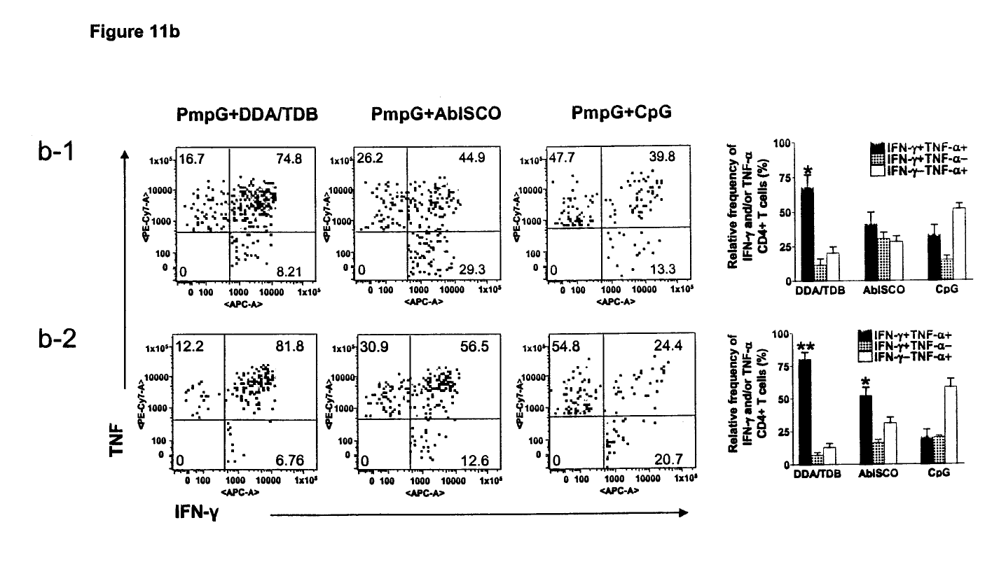

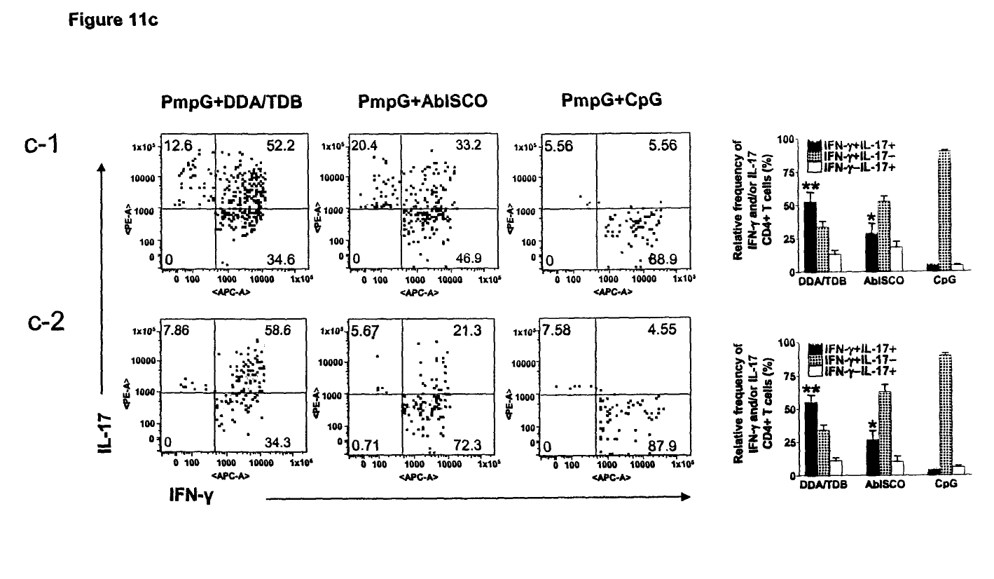

To characterize the distinct populations of Th1 and Th17 responses, multiparameter flow cytometry was used to simultaneously analyze multiple cytokines at the single-cell level. As shown in FIG. 11a, a seven-color flow cytometry panel and gating strategy was used to identify IFN-gamma, TNF-alpha and IL-17 producing CD4+ T cells in splenocytes from a representative mouse immunized with PmpG+DDA/TDB. Since an individual responding cell could be present in more than one of the total cytokine gates, we used Boolean combinations of the cytokine gates to discriminate responding cells based on their functionality or quality of IFN-γ/TNF-α (FIG. 11b) and IFN-γ/IL-17 (FIG. 11c) production.

Using the Boolean combination of IFN-γ or TNF-α gate, frequencies of three distinct populations (IFN-γ+TNF-α-, IFN-γ-TNF-α+, IFN-γ+TNF-α+) from immune splenocytes stimulated with PmpG-1 and HK-EB are shown in FIG. 11b-1 and FIG. 11b-2 respectively. The results demonstrate that the response after immunization with PmpG+DDA/TDB was dominated by IFN-γ and TNF-α double positive cells and about half of the response in the PmpG+AbISCO group was IFN-γ and TNF-α+ double positive, whereas the PmpG+CpG vaccine induced the weakest IFN-γ and TNF-α+ double positive response and the single positive dominate response. Importantly, the analysis showed a correlation between the frequency of multifunctional (IFN-γ, TNF-α double-positive) CD4+ T cells and the degree of protection in mice vaccinated with PmpG+DDA/TDB, PmpG+AbISCO and PmpG+CpG. In this study, the quality of IFN-γ/IL-17 cytokine response from immune splenocytes stimulated with PmpG (FIG. 11c-1) or HK-EB (FIG. 11c-2) was evaluated by multiparameter flow cytometry. Quantifying the fraction of IFN-γ/IL-17 response, we found that over half of the response in the most protected group (PmpG+DDA/TDB) was IFN-γ and IL-17 double positive; the PmpG+AbISCO group induced a moderate IFN-γ and IL-17 double positive response. The no protection group (PmpG+CpG) did not develop a measurable IL-17 response. The data indicate a correlation between the degree of protection in the vaccinated mice and the frequency of IFN-γ and IL-17 double positive CD4+ T cells as well as IFN-γ and TNF-α double positive CD4+ T cells.

### Example 12

### The Magnitude and Quality of IFN-γ, TNF-α and IL-17 Responses in Spleens and Lymph Nodes After Challenge

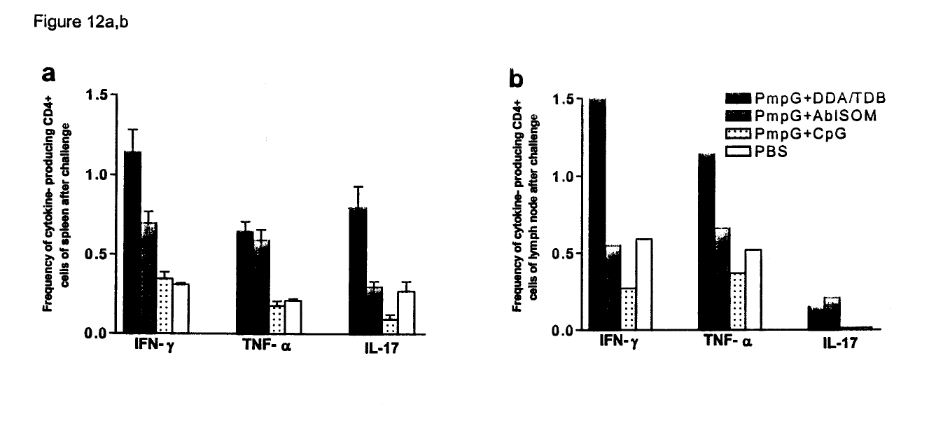

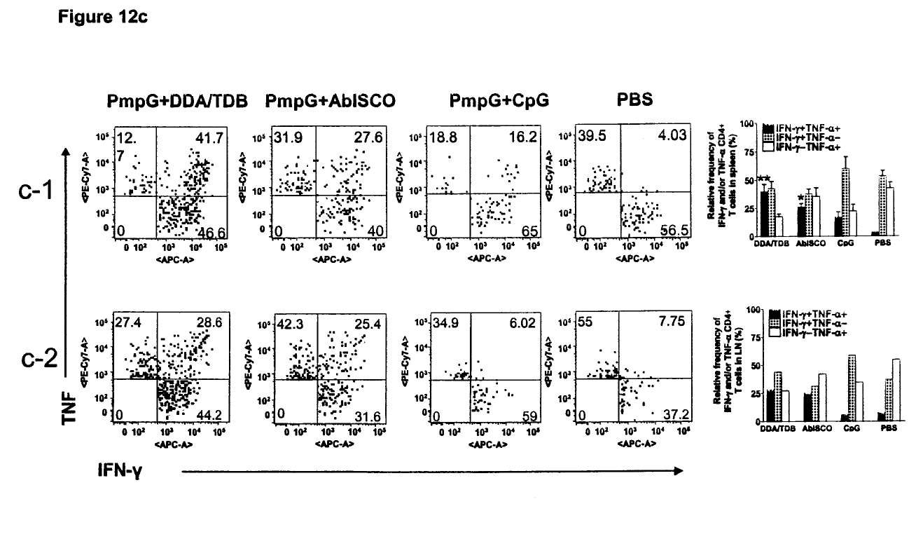

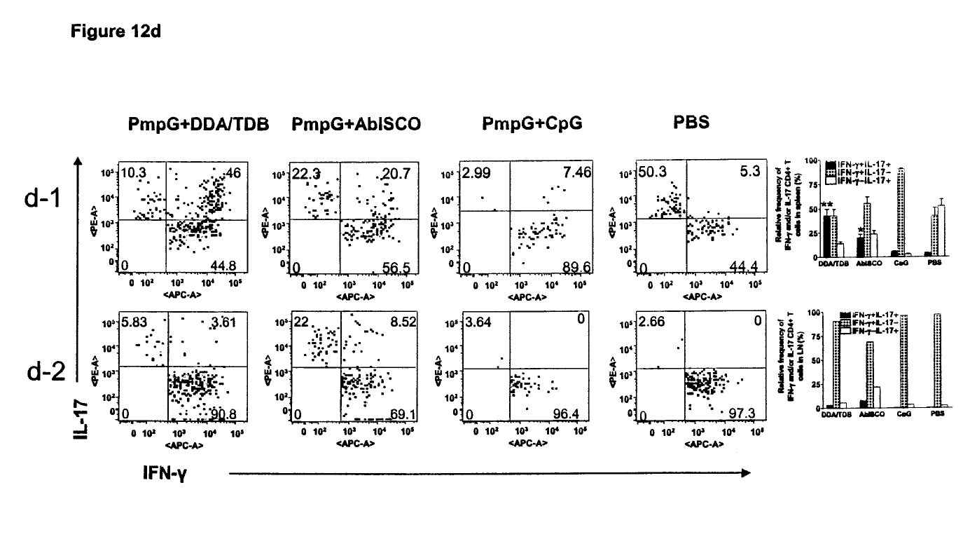

To define the magnitude of the response on day 7 after C. muridarum challenge, the frequency of the total PmpG-specific CD4+ T cell cytokine responses comprising IFN-γ, TNF-α and IL-17 producing cells in spleen (FIG. 12a) and draining lymph node (iliac lymph node) (FIG. 12b) are presented from each vaccine group. The results demonstrate among spleen cells that immunization with PmpG+DDA/TDB induced the highest frequency of IFN-γ and IL-17 producing CD4+ T cells; the PmpG+AbISCO group induced a similar frequency of TNF-α producing cell but a lower frequency of IFN-γ and IL-17 producing cells when compared with PmpG+DDA/TDB group; PmpG+CpG and PBS group developed similar but lowest frequency of IFN-γ and TNF-α producing cells. Notably, PmpG+CpG group did not induce a measurable IL-17 response while the PBS group demonstrated about one third of the magnitude for the IL-17 response compared with PmpG+DDA/TDB group (FIG. 12a). Shown is the mean±SEM (n=3 or 4) for one of at least two experiments.

The data from pooled regional draining lymph node cells following genital challenge showed that prior immunization with PmpG+DDA/TDB resulted in strong IFN-γ and TNF-α responses. The PmpG+AbISCO and PBS groups developed similar moderate IFN-γ and TNF-α responses. The PmpG+CpG group induced the weakest IFN-γ and TNF-α responses. Surprisingly, and contrary to the spleen cell results, the IL-17 response in lymph node was very low in the PmpG+DDA/TDB and PmpG+AbISCO group, and no IL-17 producing cells were observed in the PmpG+CpG and PBS group (FIG. 12b).

We further analyzed the quality of cytokine producing cells in spleen and iliac lymph node from immunized mice following genital challenge. Immunization with PmpG+DDA/TDB developed the strongest IFN-γ and TNF-α double positive response in both spleen (FIG. 12c-1) and lymph node (FIG. 12c-2). Immunization with PmpG+AbISCO induced moderate IFN-γ and TNF-α double positive response. We found very few or no IFN-γ and TNF-α double positive response cells in the PmpG+CpG group and in the PBS group (FIG. 12c-1&12c-2). Analysis of the IFN-γ/IL-17 response in spleen (FIG. 12d-1) after challenge in the three PmpG vaccine groups exhibited the strongest IFN-γ and IL-17 double positive response in PmpG+DDA/TDB group, moderate response in PmpG+AbISCO group and the weakest in PmpG+CpG. These findings show a similar pattern as before challenge (FIG. 11). However, low IL-17 producing cells, especially few IFN-γ/IL-17 double positive cells, were detected in the lymph node (FIG. 12d-2) after challenge. Notably, despite the PBS group developing IFN-γ, TNF-α and IL-17 responses after challenge, we observed that all three cytokine producing cells in this group were single positive in both spleen and lymph node (FIG. 12c and FIG. 12d). These data further confirm our findings demonstrating a connection between the level of protection and the magnitude and quality of IFN-γ, IL-17 and TNF-α production.

### Example 13

### Pathologic Changes

We evaluated the effect of immunization of the Chlamydia muridarum antigen combination on inflammatory pathology in C57BL/6 mouse upper genital tract following Chlamydia muridarum infection. Sixty days after the intravaginal challenge infection, mice were sacrificed and mouse genital tract tissues were collected for pathology observation. The genital tract tissues from mice immunized with G+F+M+DDA/TDB, G+F+M+AbISCO, PBS or live EB were examined at the level of gross appearance (G+F+M=PmpG-1, PmpE/F-2 and MOMP pooled). Hydrosalpinx is a visual hallmark of inflammatory pathology in the fallopian tube induced by Chlamydia muridarum infection. Six of 8 mice in PBS group developed obvious hydrosalpinx in either one or both fallopian tubes (3 mice bilateral, 3 mice unilateral). Six of the 8 mice vaccinated with G+F+M+DDA/TDB (2 mice bilateral, 4 mice unilateral) and eight of the 8 mice vaccinated with G+F+M+AbISCO (4 mice bilateral, 4 mice unilateral) had hydrosalpinx. The pathologic outcome in both G+F+M+DDA/TDB and G+F+M+AbISCO groups was not significantly different from that in PBS group. Mice recovered from a prior intranasal infection were however completely protected against the development of oviductal hydrosalpinx pathology.

### Example 14

### Induction of CD4+ T Cells by C. trachomatis Epitopes

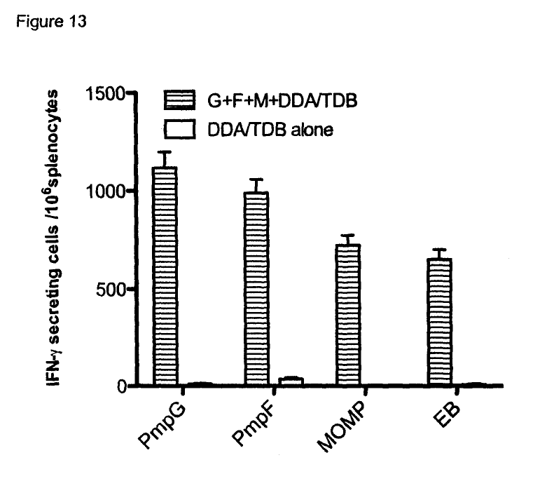

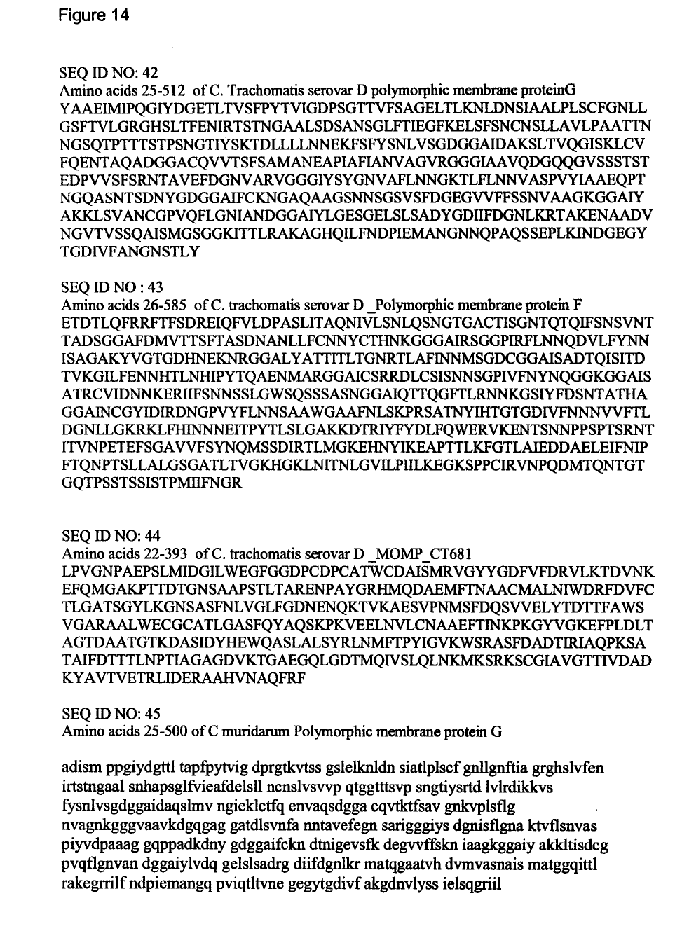

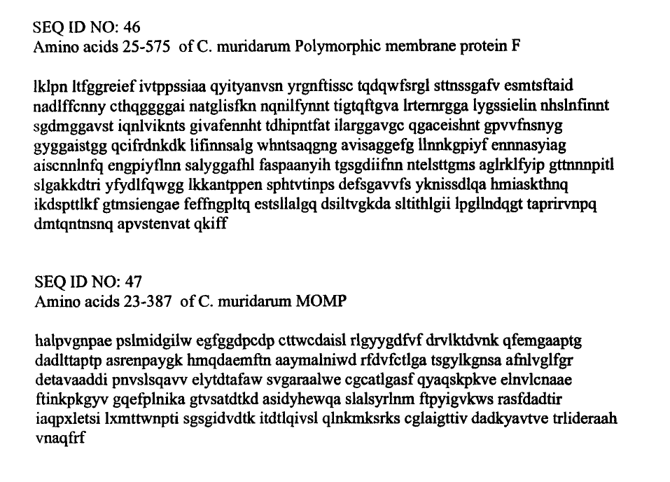

C57 BL/6 mice were immunized three times subcutaneously in the base of tail with a cocktail of C. trachomatis serovar D polypeptides PmpG (SEQ ID NO: 42), PmpF (SEQ IDN O: 43) and MOMP (SEQ ID NO: 44), formulated with DDA/TDB adjuvant (G+F+M+DDA/TDB) at 2-week intervals. Adjuvant alone (DDA/TDB) was administered as control. Two weeks after the final immunization, splenocytes were harvested and stimulated with 1 microgram/ml C. trachomatis serovar D protein PmpG, PmpF, MOMP or 5×10⁵ inclusion-forming units (IFU)/ml heat-killed EB respectively. DDA/TDB alone adjuvant was set up as a negative control. Interferon gamma response in mice was determined by an ELISPOT assay (FIG. 13). The results represent the average of duplicate wells and are expressed as means±SEM for groups of six mice.

These studies demonstrate that CD4+ T cells of mice immunized with a C. trachomatis antigen composition can be stimulated by individual components of the antigen composition and produce IFN-gamma.

All citations are herein incorporated by reference, as if each individual publication was specifically and individually indicated to be incorporated by reference herein and as though it were fully set forth herein. Citation of references herein is not to be construed nor considered as an admission that such references are prior art to the present invention.

One or more currently preferred embodiments of the invention have been described by way of example. The invention includes all embodiments, modifications and variations substantially as hereinbefore described and with reference to the examples and figures.

It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

What is claimed is:

1. A composition for inducing an immune response to a Chlamydia species in a subject, the composition comprising one, or more than one, polypeptide selected from the group consisting of SEQ ID NO: 10, SEQ ID NO: 13, SEQ ID NO: 16, SEQ ID NO:17, SEQ ID NO: 20, SEQ ID NO: 21, SEQ ID NO: 22, SEQ ID NO: 23, SEQ ID NO: 27, SEQ ID NO: 28, SEQ ID NO: 31, SEQ ID NO: 42, SEQ ID NO: 43, SEQ ID NO: 45 and SEQ ID NO: 46, and an excipient.

2. The composition of claim 1 wherein the polypeptides are Chlamydia trachomatis polypeptides, or Chlamydia muridarum polypeptides.

3. The composition of claim 1 further comprising an adjuvant.

4. The composition of claim 1, further comprising a MOMP polypeptide according to SEQ ID NO: 44 or SEQ ID NO: 47.

5. The composition of claim 3, wherein the adjuvant is dimethyldioctadecylammonium bromide and trehalose 6,6′-dibehenate (DDA/TDB) or AbISCO.

6. The composition of claim 1 wherein the immune response is a cellular immune response.

7. The composition of claim 1 wherein the Chlamydia species is C. trachomatis or C. muridarum.

8. A method of treating or preventing a Chlamydia infection in a subject, comprising administering to the subject an effective amount of a composition comprising one or more than one polypeptides selected from the group consisting of SEQ ID NO: 10, SEQ ID NO: 13, SEQ ID NO: 16, SEQ ID NO:17, SEQ ID NO: 20, SEQ ID NO: 21, SEQ ID NO: 22, SEQ ID NO: 23, SEQ ID NO: 27, SEQ ID NO: 28, SEQ ID NO: 31, SEQ ID NO: 42, SEQ ID NO: 43, SEQ ID NO: 45, SEQ ID NO: 46, PmpG, PmpF, PmpG-1, PmpE/F-2 and RplF, and an excipient.

9. The method of claim 8 wherein the Chlamydia infection is in a lung or genital tract.

10. The method of claim 8 wherein the composition induces a cellular immune response.

11. The method of claim 8 wherein the Chlamydia infection is associated with C. trachomatis.

12. The method of claim 8 wherein the composition is administered intranasally, or is injected.

13. A composition for inducing an immune response in a subject, comprising one or more polypeptides selected from the group consisting of SEQ ID NO: 10, SEQ ID NO: 13, SEQ ID NO: 16, SEQ ID NO:17, SEQ ID NO: 20, SEQ ID NO: 21, SEQ ID NO: 22, SEQ ID NO: 23, SEQ ID NO: 27, SEQ ID NO: 28, SEQ ID NO: 31, SEQ ID NO: 42, SEQ ID NO: 43, SEQ ID NO: 45, SEQ ID NO: 46, PmpG, PmpF, PmpG-1, PmpE/F-2 and RplF, and an excipient.

14. The composition of claim 13 wherein the polypeptides PmpG, PmpF, PmpG-1, PmpE/F-1 or RplF are Chlamydia trachomatis polypeptides, or Chlamydia muridarum polypeptides.

15. A method of eliciting an immune response against Chlamydia trachomatis in a mammal, comprising administration of a therapeutically effective amount of a composition comprising one or more C. trachomatis polypeptides and an excipient.

16. Use of the composition of claim 1 for treatment or prevention of a Chlamydia infection in a subject.

17. Use of the composition of claim 1 in the manufacture of a medicament for treatment or prevention of a Chlamydia infection in a subject.

18. A method of treating or preventing a Chlamydia infection comprising administering an effective amount of a composition of claim 1.

19. The method of claim 15, wherein the one or more C. trachomatis polypeptides are selected from the group consisting of PmpG, PmpF, SEQ ID NO: 43, SEQ ID NO: 44 and RplF.

20. A composition comprising one or more than one of PmpG (SEQ ID NO: 42), PmpF (SEQ ID NO: 43) and MOMP (SEQ ID NO: 44) of C. trachomatis, and dimethyldioctadecylammonium bromide and trehalose 6,6′-dibehenate (DDA/TDB).

21. The composition of claim 1, further comprising one, or more than one, of a polypeptide selected from the group consisting of PmpG, PmpF, PmpG-1, PmpE/F-2, and RplF.

22. The composition of claim 21, wherein the polypeptides are Chlamydia trachomatis polypeptides, or Chlamydia muridarum polypeptides.


SEQUENCE LISTING (48 sequences)

SEQ ID NO: 1-9 - skipped (9 entries)

SEQ ID NO: 10 - protein, 17 aa, Artificial sequence
  Ribosomal protein L/Rplf
GNEVFVSPAAHIIDRPG

SEQ ID NO: 11 - protein, 18 aa, Artificial sequence
  ACP reductase
SPGQTNYAAAKAGIIGFS

SEQ ID NO: 12 - protein, 16 aa, Artificial sequence
  Anti-anti-sigma factor
KLDGVSSPAVQESISE

SEQ ID NO: 13 - protein, 17 aa, Artificial sequence
  Polymorphic membrane protein G
ASPIYVDPAAAGGQPPA

SEQ ID NO: 14 - protein, 14 aa, Artificial sequence
  Hypothetical protein
DLNVTGPKIQTDVD

SEQ ID NO: 15 - protein, 15 aa, Artificial sequence
  ATP-dependent Clp protease proteolytic subunit
IGQEITEPLANTVIA

SEQ ID NO: 16 - protein, 16 aa, Artificial sequence
  Polymorphic membrane protein F
AFHLFASPAANYIHTG

SEQ ID NO: 17 - protein, 15 aa, Artificial sequence
  Glyceraldehyde 3-phosphate dehydrogenase
MTTVHAATATQSVVD

SEQ ID NO: 18 - skipped

SEQ ID NO: 19 - protein, 8 aa, C. trachomatis
SSLFLVKL

SEQ ID NO: 20 - protein, 14 aa, C. trachomatis
GNEVFVSPAAHIID

SEQ ID NO: 21 - protein, 17 aa, C. trachomatis
GNEVFVSPAAHIIDRPG

SEQ ID NO: 22 - protein, 18 aa, C. trachomatis
KGNEVFVSPAAHIIDRPG

SEQ ID NO: 23 - protein, 15 aa, C. trachomatis
EVFVSPAAHIIDRPG

SEQ ID NO: 24 - protein, 16 aa, C. trachomatis
SPGQTNYAAAKAGIIG

SEQ ID NO: 25 - protein, 18 aa, C. trachomatis
SPGQTNYAAAKAGIIGFS

SEQ ID NO: 26 - protein, 16 aa, C. trachomatis
KLDGVSSPAVQESISE

SEQ ID NO: 27 - protein, 16 aa, C. trachomatis
SPIYVDPAAAGGQPPA

SEQ ID NO: 28 - protein, 17 aa, C. trachomatis
ASPIYVDPAAAGGQPPA

SEQ ID NO: 29 - protein, 14 aa, C. trachomatis
DLNVTGPKIQTDVD

SEQ ID NO: 30 - protein, 15 aa, C. trachomatis
IGQEITEPLANTVIA

SEQ ID NO: 31 - protein, 16 aa, C. trachomatis
FHLFASPAANYIHTGP

SEQ ID NO: 32 - protein, 15 aa, C. trachomatis
MTTVHAATATQSVVD

SEQ ID NO: 33-41 - skipped (9 entries)

SEQ ID NO: 42 - protein, 487 aa, C. trachomatis
YAAEIMIPQGIYDGETLTVSFPYTVIGDPSGTTVFSAGELTLKNLDNSIAALPLSCFGNLLGSFTVLGRGHSLTFENIRTSTNGAALSDSANSGLFTIEGFKELSFSNCNSLLAVLPAATTNNGSQTPTTTSTPSNGTIYSKTDLLLLNNEKFSFYSNLVSGDGGAIDAKSLTVQGISKLCVFQENTAQADGGACQVVTSFSAMANEAPIAFIANVAGVRGGGIAAVQDGQQGVSSSTSTEDPVVSFSRNTAVEFDGNVARVGGGIYSYGNVAFLNNGKTLFLNNVASPVYIAAEQPTNGQASNTSDNYGDGGAIFCKNGAQAAGSNNSGSVSFDGEGVVFFSSNVAAGKGGAIYAKKLSVANCGPVQFLGNIANDGGAIYLGESGELSLSADYGDIIFDGNLKRTAKENAADVNGVTVSSQAISMGSGGKITTLRAKAGHQILFNDPIEMANGNNQPAQSSEPLKINDGEGYTGDIVFANGNSTLY

SEQ ID NO: 43 - protein, 559 aa, C. trachomatis
ETDTLQFRRFTFSDREIQFVLDPASLITAQNIVLSNLQSNGTGACTISGNTQTQIFSNSVNTTADSGGAFDMVTTSFTASDNANLLFCNNYCTHNKGGGAIRSGGPIRFLNNQDVLFYNNISAGAKYVGTGDHNEKNRGGALYATTITLTGNRTLAFINNMSGDCGGAISADTQISITDTVKGILFENNHTLNHIPYTQAENMARGGAICSRRDLCSISNNSGPIVFNYNQGGKGGAISATRCVIDNNKERIIFSNNSSLGWSQSSSASNGGAIQTTQGFTLRNNKGSIYFDSNTATHAGGAINCGYIDIRDNGPVYFLNNSAAWGAAFNLSKPRSATNYIHTGTGDIVFNNNVVFTLDGNLLGKRKLFHINNNEITPYTLSLGAKKDTRIYFYDLFQWERVKENTSNNPPSPTSRNTITVNPETEFSGAVVFSYNQMSSDIRTLMGKEHNYIKEAPTTLKFGTLAIEDDAELEIFNIPFTQNPTSLLALGSGATLTVGKHGKLNITNLGVILPIILKEGKSPPCIRVNPQDMTQNTGTGQTPSSTSSISTPMIIFNGR

SEQ ID NO: 44 - protein, 371 aa, C. trachomatis
LPVGNPAEPSLMIDGILWEGFGGDPCDPCATWCDAISMRVGYYGDFVFDRVLKTDVNKEFQMGAKPTTDTGNSAAPSTLTARENPAYGRHMQDAEMFTNAACMALNIWDRFDVFCTLGATSGYLKGNSASFNLVGLFGDNENQKTVKAESVPNMSFDQSVVELYTDTTFAWSVGARAALWECGCATLGASFQYAQSKPKVEELNVLCNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNKMKSRKSCGIAVGTTIVDADKYAVTVETRLIDERAAHVNAQFRF

SEQ ID NO: 45 - protein, 475 aa, C. muridarum
ADISMPPGIYDGTTLTAPFPYTVIGDPRGTKVTSSGSLELKNLDNSIATLPLSCFGNLLGNFTIAGRGHSLVFENIRTSTNGAALSNHAPSGLFVIEAFDELSLLNCNSLVSVVPQTGGTTTSVPSNGTIYSRTDLVLRDIKKVSFYSNLVSGDGGAIDAQSLMVNGIEKLCTFQENVAQSDGGACQVTKTFSAVGNKVPLSFLGNVAGNKGGGVAAVKDGQGAGGATDLSVNFANNTAVEFEGNSARIGGGIYSDGNISFLGNAKTVFLSNVASPIYVDPAAAGGQPPADKDNYGDGGAIFCKNDTNIGEVSFKDEGVVFFSKNIAAGKGGAIYAKKLTISDCGPVQFLGNVANDGGAIYLVDQGELSLSADRGDIIFDGNLKRMATQGAATVHDVMVASNAISMATGGQITTLRAKEGRRILFNDPIEMANGQPVIQTLTVNEGEGYTGDIVFAKGDNVLYSSIELSQGRIIL

SEQ ID NO: 46 - protein, 550 aa, C. muridarum
LKLPNLTFGGREIEFIVTPPSSIAAQYITYANVSNYRGNFTISSCTQDQWFSRGLSTTNSSGAFVESMTSFTAIDNADLFFCNNYCTHQGGGGAINATGLISFKNNQNILFYNNTTIGTQFTGVALRTERNRGGALYGSSIELINNHSLNFINNTSGDMGGAVSTIQNLVIKNTSGIVAFENNHTTDHIPNTFATILARGGAVGCQGACEISHNTGPVVFNSNYGGYGGAISTGGQCIFRDNKDKLIFINNSALGWHNTSAQGNGAVISAGGEFGLLNNKGPIYFENNNASYIAGAISCNNLNFQENGPIYFLNNSALYGGAFHLFASPAANYIHTGSGDIIFNNNTELSTTGMSAGLRKLFYIPGTTNNNPITLSLGAKKDTRIYFYDLFQWGGLKKANTPPENSPHTVTINPSDEFSGAVVFSYKNISSDLQAHMIASKTHNQIKDSPTTLKFGTMSIENGAEFEFFNGPLTQESTSLLALGQDSILTVGKDASLTITHLGIILPGLLNDQGTTAPRIRVNPQDMTQNTNSNQAPVSTENVATQKIFF

SEQ ID NO: 47 - protein, 367 aa, C. Muridarum
  misc_feature (285)..(285): Xaa can be any naturally occurring amino acid
  misc_feature (292)..(292): Xaa can be any naturally occurring amino acid
HALPVGNPAEPSLMIDGILWEGFGGDPCDPCTTWCDAISLRLGYYGDFVFDRVLKTDVNKQFEMGAAPTGDADLTTAPTPASRENPAYGKHMQDAEMFTNAAYMALNIWDRFDVFCTLGATSGYLKGNSAAFNLVGLFGRDETAVAADDIPNVSLSQAVVELYTDTAFAWSVGARAALWECGCATLGASFQYAQSKPKVEELNVLCNAAEFTINKPKGYVGQEFPLNIKAGTVSATDTKDASIDYHEWQASLALSYRLNMFTPYIGVKWSRASFDADTIRIAQPXLETSILXMTTWNPTISGSGIDVDTKITDTLQIVSLQLNKMKSRKSCGLAIGTTIVDADKYAVTVETRLIDERAAHVNAQFRF

SEQ ID NO: 48 - DNA, 20 bp, Artificial Sequence
  CpG ODN
tccatgacgttcctgacgtt